(19) United States
(12) Patent Application Publication
(10) Pub. No.: US 2013/0295590 A1
(43) Pub. Date: Nov. 7, 2013

(54) FOXA1 AS A MARKER FOR INVASIVE BLADDER CANCER

(71) Applicants: David Degraff, Nashville, TN (US); Robert Matusik, Nashville, TN (US); Peter Clark, Nashville, TN (US); Dan Theodorescu, Aurora, CO (US)

(72) Inventors: David Degraff, Nashville, TN (US); Robert Matusik, Nashville, TN (US); Peter Clark, Nashville, TN (US); Dan Theodorescu, Aurora, CO (US)

(73) Assignees: The Regents of the University of Colorado, A body Corporate, Denver, CO (US); VANDERBILT UNIVERSITY, Nashville, TN (US)

(21) Appl. No.: 13/885,376

(22) PCT Filed: Nov. 8, 2011
(86) PCT No.: PCT/US11/59762

§ 371 Date: Jul. 26, 2013

Related U.S. Application Data
(60) Provisional application No. 61/413,877, filed on Nov. 15, 2010.

(51) Int. Cl. G01N 33/68

(52) U.S. Cl. CPC G01N 33/6893
USPC 435/7.92; 436/501; 435/7.1; 435/23

(57) ABSTRACT

The present invention provides for a proteomic approach to predicting, diagnosing and staging invasive bladder cancer, and for predicting patient survival and therapeutic efficacy. More specifically, the target being analyzed for reduced expression is FOXA1, and optionally including analysis of increased FOXA2 expression.

This application claims benefit of priority to U.S. Provisional Application Ser. No. 61/413,877, filed Nov. 15, 2010, the entire contents of which are hereby incorporated by reference.

### BACKGROUND OF THE INVENTION

1. Field of the Invention

The invention relates to the fields of molecular and cellular biology, particularly to the detection of FoxA1 as a diagnostic and prognostic factor for bladder cancer.

2. Description of Related Art

Bladder cancer is the fifth most common cancer in the United States accounting for about 4.6% of all cases (Jemal et al., 2007). Most incidences of bladder cancer are superficial and localized in character with about 74% of all cases being localized when diagnosed (Jemal et al., 2007). It exists in two main forms, non-invasive which lacks invasion into surrounding muscle tissue and is the more common form accounting for 75% of all cases and muscle invasive in which it spreads into surrounding urinary areas and may metastasize (Sengupta and Blute, 2006).

The cost for treating bladder cancer is considerable due to lifetime surveillance, treatment of recurrent disease, and costs of complications associated with treatments. One estimate puts lifetime costs at between $100K-$125K, with an estimated cost to Medicare over the next five years of $1,000,000,000.

When diagnosed at early stages bladder cancer has about a 94% survival rate, but this rate drops dramatically to 46% when the cancer has spread to the surrounding region and to 6% when the cancer is distant at diagnosis (Jemal et al., 2007). Although most forms are non-invasive they have a large risk of recurrence after treatment (>50%) and high-grade superficial lesions carry a significant risk of progression (Sengupta and Blute, 2006).

The standard method of diagnosing bladder cancer is visualization by cystoscopy (Clark, 2007). However this technique is invasive, costly, and is limited by needing sight interpretations of growths (Sengupta and Blute, 2006). Inspection of cells in urine for abnormalities (urine cytology) and detection of chromosomal abnormalities in those cells (FISH or fluorescence in situ hybridization) are two other common methods of diagnosis without the invasiveness of cytology (Sengupta and Blute, 2006; Clark, 2007). However, urine cytology has very low sensitivity in detection of low grade disease (Sengupta and Blute, 2006; Clark, 2007) and FISH, while better at detecting low grade forms still carries low specificity and sensitivity and is subject to wide variation in effectiveness among cases (Clark, 2007).

More sensitive and specific early detection tools for bladder cancer would greatly improve patient survival. Also given the high recurrence rate, an easy follow up procedure that could be administered regularly would greatly improve treatment of recurrences. Urine, semen and prostatic fluid analysis, these fluids having come into close contact with genitourinary tissues, also would provide a tool unique to this kind of cancer. Advantageously, such procedures would be non-invasive and easy to conduct.

### SUMMARY OF THE INVENTION

Thus, in accordance with the present invention, there is provided a method of predicting or diagnosing invasive bladder cancer in a sample from a subject comprising assessing reduced FoxA1 expression, as compared to normal controls Assessing may comprise immunologic detection, or mass spectrometry, such as secondary ion mass spectrometry, laser desorption mass spectrometry, matrix assisted laser desorption mass spectrometry, or electrospray mass spectrometry, or detecting expression of a FoxA1 transcript, such as after amplifying said transcript or a nucleic acid derived therefrom, such as by RT-PCR. The sample may be urine, semen or prostatic fluid from said subject, or a tumor tissue sample from said subject.

Where the method is immunologic, assessing may further comprise exposing said sample to a first anti-FoxA1 antibody and a second anti-FoxA1 antibody, said first and second anti-FoxA1 antibodies binding to different epitopes. The second anti-FoxA1 antibody may comprise a detectable label, or the second anti-FoxA1 may be detected using an anti-Fc antibody that is labeled with a detectable marker. The first anti-FoxA1 antibody is affixed to a support such a membrane, a dipstick, a multi-well dish, a filter, a bead, or a biochip.

The invasive bladder cancer may be muscle-invasive and/or metastatic bladder cancer. The subject may previously have been diagnosed with bladder cancer and as was successfully treated for said bladder cancer. The assessing may comprise a lateral flow ELISA assay. The method may further comprise assessing one or more patient variables, such as age, gender, extent of tumor resection, use of pre-surgery chemotherapy, or use of pre-surgery radiotherapy.

In another embodiment, there is provided a method of monitoring the progression and/or prognosing of invasive bladder cancer in a subject comprising assessing FoxA1 level in the urine, semen or prostatic fluid of said subject at multiple time points, wherein an decrease in FoxA1 level over time indicates progression of said bladder cancer and a poor prognosis. Assessing may comprise immunologic detection, or mass spectrometry, such as secondary ion mass spectrometry, laser desorption mass spectrometry, matrix assisted laser desorption mass spectrometry, or electrospray mass spectrometry, or detecting expression of a FoxA1 transcript, such as after amplifying said transcript or a nucleic acid derived therefrom, such as by RT-PCR. The sample may be urine, semen or prostatic fluid from said subject, or a tumor tissue sample from said subject.

Where the method is immunologic, assessing may further comprise exposing said sample to a first anti-FoxA1 antibody and a second anti-FoxA1 antibody, said first and second anti-FoxA1 antibodies binding to different epitopes. The second anti-FoxA1 antibody may comprise a detectable label, or the second anti-FoxA1 may be detected using an anti-Fc antibody that is labeled with a detectable marker. The first anti-FoxA1 antibody is affixed to a support such a membrane, a dipstick, a multi-well dish, a filter, a bead, or a biochip.

The invasive bladder cancer may be muscle-invasive or metastatic bladder cancer. The multiple time points may be separated by at least one month and/or increase with time. The assessing may comprise a lateral flow ELISA assay. The method may further comprise assessing one or more patient variables, such as age, gender, extent of tumor resection, use of pre-surgery chemotherapy, or use of pre-surgery radiotherapy.

In yet another embodiment, there is provided a method of monitoring the treatment of bladder cancer in a subject comprising assessing a FoxA1 level in the urine, semen or prostatic fluid of said subject at multiple time points, wherein an increase in FoxA1 level over time indicates treatment efficacy. The method may further comprise altering a treatment plan based on the FoxA1 level. Assessing may comprise immunologic detection, or mass spectrometry, such as secondary ion mass spectrometry, laser desorption mass spectrometry, matrix assisted laser desorption mass spectrometry, or electrospray mass spectrometry, or detecting expression of a FoxA1 transcript, such as after amplifying said transcript or a nucleic acid derived therefrom, such as by RT-PCR. The sample may be urine, semen or prostatic fluid from said subject, or a tumor tissue sample from said subject.

Where the method is immunologic, assessing may further comprise exposing said sample to a first anti-FoxA1 antibody and a second anti-FoxA1 antibody, said first and second anti-FoxA1 antibodies binding to different epitopes. The second anti-FoxA1 antibody may comprise a detectable label, or the second anti-FoxA1 may be detected using an anti-Fc antibody that is labeled with a detectable marker. The first anti-FoxA1 antibody is affixed to a support such a membrane, a dipstick, a multi-well dish, a filter, a bead, or a biochip.

The bladder cancer may be invasive bladder cancer, muscle-invasive bladder cancer, superficial bladder cancer or metastatic bladder cancer. The multiple time points may be separated by at least one month and/or increase with time. The assessing may comprise a lateral flow ELISA assay. The method may further comprise assessing one or more patient variables, such as age, gender, extent of tumor resection, use of pre-surgery chemotherapy, or use of pre-surgery radiotherapy.

In still another embodiment, there is provided a method of staging a bladder cancer in a subject comprising assessing FoxA1 level in the urine, semen or prostatic fluid of said subject and comparing said level to a predetermined level for one or more given stages of a genitourinary cancer. The bladder cancer may be Stage 0, Stage I, Stage II, Stage III, Stage IV, or recurrent. The method may further comprise altering a treatment plan based on the FoxA1 level.

Detecting may comprise immunologic detection, or mass spectrometry, such as secondary ion mass spectrometry, laser desorption mass spectrometry, matrix assisted laser desorption mass spectrometry, or electrospray mass spectrometry, or detecting expression of a FoxA1 transcript, such as after amplifying said transcript or a nucleic acid derived therefrom, such as by RT-PCR. The sample may be urine, semen or prostatic fluid from said subject, or a tumor tissue sample from said subject.

Where the method is immunologic, detection may further comprises exposing said sample to a first anti-FoxA1 antibody and a second anti-FoxA1 antibody, said first and second anti-FoxA1 antibodies binding to different epitopes. The second anti-FoxA1 antibody may comprise a detectable label, or the second anti-FoxA1 may be detected using an anti-Fc antibody that is labeled with a detectable marker. The first anti-FoxA1 antibody is affixed to a support such a membrane, a dipstick, a multi-well dish, a filter, a bead, or a biochip.

The bladder cancer may be invasive bladder cancer, muscle-invasive superficial bladder cancer or metastatic bladder cancer. The detection may comprise a lateral flow ELISA assay. The method may further comprise assessing one or more patient variables, such as age, gender, extent of tumor resection, use of pre-surgery chemotherapy, or use of pre-surgery radiotherapy.

In still a further embodiment, there is provided a method of predicting response of bladder cancer in a subject to a chemo- or radiotherapy comprising assessing FoxA1 level in the urine, semen or prostatic fluid of said subject at multiple time points, wherein the lower the FoxA1 level, better chance of therapeutic efficacy.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed in this specification can be implemented with respect to any method or composition of the invention, and vice versa. Furthermore, compositions and kits of the invention can be used to achieve methods of the invention.

Throughout this application, the term “about” is used to indicate that a value includes the inherent variation of error for the device, the method being employed to determine the value, or the variation that exists among the study subjects.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-B: FOXA1 expression correlates with uroplakin expression in vitro and in human bladder tissue. (FIG. 1A) A panel of commonly used urothelial cell lines was screened via traditional RT-PCR for the presence of FOXA1, FOXA2, and FOXA3 transcripts, as well as members of the uroplakin (UPK) family. HepG2 cells, which express each member of the FOXA subfamily, were used as positive controls (data not shown). RT4 cells exhibited robust expression of FOXA1 and as previously reported, UPK family members. FOXA2 expression was detected in T24 cells, but was not correlated with UPK family member expression. FOXA3 was not detected in any tested cell line (data not shown). (FIG. 1B) Decreased FOXA1 expression is associated with decreased UPK expression in human tissue. Archival normal adjacent tissue (top panel) and bladder tumor was immunostained with a pan-UPK antibody, AUM as well as an antibody directed against FOXA1. Representative cases are illustrated. Results suggest that decreased FOXA1 is associated with decreased UPK expression in human urothelial carcinoma cell lines and human tumor samples.

FIGS. 2A-B: FOXA1 expression is lost in most high grade, advanced stage muscle invasive bladder cancers. (FIG. 2A) Serial sections of AJCC stage Tis, Ta, T1, T2, T3 and T4 bladder tumors were immunostained for H&E, FOXA1 and FOXA2. Representative cases are illustrated. (FIG. 2B) Loss of FOXA1 staining was observed with advanced tumor stage (p<0.001). A small subset of invasive tumors exhibited nuclear expression of FOXA2.

FIGS. 3A-F: FOXA1 expression is lost in a subset of lymph node metastases. H&E (FIGS. 3A, 3C, and 3E) of FOXA1-positive (FIG. 3B) and FOXA1-negative (FIGS. 3D and 3F) metastatic lymph node samples isolated from bladder cancer patients are depicted. Transitional (urothelial) cell carcinoma (FIGS. 3A and 3B) and squamous cell carcinomas (FIGS. 3C-D and 3E-F) are shown.

FIG. 4: FOXA1 expression is absent in keratinizing squamous metaplasia of bladder urothelium, a recognized precursor to development of squamous cell carcinoma.

FIGS. 5A-D: Decreased FOXA1 expression in squamous cell carcinoma (SCC) of the urinary bladder. H&E (FIGS. 5A and 5C) of FOXA1-positive (FIG. 5B) and FOXA1-negative (FIG. 5D) samples of human SCC of the urinary bladder are depicted. Most cases (81%) of bladder SCC showed loss of FOXA1 expression.

FIGS. 6A-C: FOXA1 Knock-down results in increased proliferation of RT4 tissue recombinants. (FIG. 6A) FOXA1 expression in well-differentiated RT4 human bladder cancer cells decreased via stable expression of shRNA under puromycin selection. RT4 cells stably expressing scrambled construct or FOXA1-specific shRNA were recombined with bladder mesenchyme isolated from embryonic-16 day old rats and inserted under the kidney capsule of immunocompromised mice. After three weeks, host mice were injected with BRDU and sacrificed. (FIG. 6B) Tumor volume was increased in FOXA1 KD RT4 cells, and RT4-FOXA1 knock down cells showed increased incorporation of BRDU (FIG. 6C), indicating FOXA1 knock down results in increased bladder cancer cell proliferation.

FIG. 7: Changes in FOXA1 expression results in cell-line specific alterations in oncogene expression and cell signaling. RT4 cells were engineered to express diminished FOXA1 expression (RT4 FOXA1 KD, left panel), and T24 cells were engineered to ectopically overexpress FOXA1 (T24 FOXA10E, right panel). Western blotting analysis showed overexpression of FOXA1 resulted in increased expression of EGFR, increased phosphorylation of AKT, and increased expression of the metastasis suppressor, E-cadherin. FOXA1 knock down resulted in decreased phosphorylation of FAK and decreased expression of E-cadherin. These results indicate FOXA1 tumor status may be useful in identifying patients who would respond to EGFR blockade and/or retinoic acid/TZD treatment.

FIG. 8: Bladder cancer staging.

### DESCRIPTION OF ILLUSTRATIVE EMBODIMENTS

Approximately 50% of patients diagnosed with muscle-invasive bladder cancer (MIBC) develop metastatic disease, which is almost invariably lethal. Therefore, identification of pathways that drive the aggressive behavior of MIBC and/or predict patient outcome are of paramount importance. Aberrations within pathways that dictate normal organogenesis and differentiation are theorized to be critical events during tumor initiation and progression. Recent studies have implicated members of the Forkhead Box A (FOXA) subfamily of transcription factors, namely FOXA1, in expression of urothelial-specific uroplakin (UPK) proteins and urothelial differentiation. However, the role of FOXA proteins in bladder cancer is unknown. Therefore, the inventors examined FOXA family member expression in commonly used in vitro models of bladder cancer and in human bladder cancer specimens.

The inventors report FOXA1 and FOXA2 expression in specific bladder cancer cell lines, and that UPK expression was correlated with FOXA1 expression in human bladder cancer cell lines and human bladder tumors. In addition, while FOXA1 is uniformly expressed in early stage (non-muscle-invasive) bladder tumors, its expression is significantly decreased in MIBCl, although it was expressed in a small number of MIBC samples. Further, the absence of FOXA1 staining was correlated to squamous cell carcinoma (SCC) of the bladder were negative for FOXA1 staining, while more transitional cell carcinomas (TCC) showed FOXA1. FOXA1 expression was also decreased in keratinizing squamous metaplasia, a recognized precursor to SCC. The inventors also report that decreased FOXA1 expression is associated with muscle invasion, high histologic grade, female gender and metastatic tumor deposits in lymph nodes. These findings support a role for FOXA1 in urothelial differentiation and provide the first evidence linking loss of FOXA1 expression with SCC and TCC subtypes of MIBC.

### I. BLADDER CANCER

A. General Background

Bladder cancer refers to any of several types of malignant growths of the urinary bladder, with over 65,000 new cases and some 13,750 attributed deaths reported in 2007 alone. It is a disease in which abnormal cells multiply without control in the bladder. The bladder is a hollow, muscular organ that stores urine; it is located in the pelvis. The most common type of bladder cancer begins in cells lining the inside of the bladder and is called urothelial cell or transitional cell carcinoma (UCC or TCC).

Bladder cancer characteristically causes blood in the urine, this may be visible to the naked eye (frank haematuria) or detectable only be microscope (microscopic haematuria). Other possible symptoms include pain during urination, frequent urination or feeling the need to urinate without results. These signs and symptoms are not specific to bladder cancer, and are also caused by non-cancerous conditions, including prostate infections and cystitis.

Exposure to environmental carcinogens of various types is responsible for the development of most bladder cancers. Tobacco use (specifically cigarette smoking) is thought to cause 50% of bladder cancers discovered in male patients and 30% of those found in female patients. Thirty percent of bladder tumors probably result from occupational exposure in the workplace to carcinogens such as benzidine. Occupations at risk are metal industry workers, rubber industry workers, workers in the textile industry and people who work in printing. Hairdressers are thought to be at risk as well because of their frequent exposure to permanent hair dyes. It has been proposed that hair dyes are a risk factor, and some have shown an odds ratio of 2.1 to 3.3 for risk of developing bladder cancer among women who use permanent hair yes, while others have shown no correlation between the use of hair dyes and bladder cancer. Certain drugs such as cyclophosphamide and phenacetin are known to predispose to bladder TCC. Chronic bladder irritation (infection, bladder stones, catheters, bilharzia) predisposes to squamous cell carcinoma of the bladder. Approximately 20% of bladder cancers occur in patients without predisposing risk factors. Bladder cancer is not currently believed to be heritable.

Like virtually all cancers, bladder cancer development involves the acquisition of mutations in various oncogenes and tumor suppressor genes. Genes which may be altered in bladder cancer include FGFR3, HRAS, RB1 and P53. Several genes have been identified which play a role in regulating the cycle of cell division, preventing cells from dividing too rapidly or in an uncontrolled way. Alterations in these genes may help explain why some bladder cancers grow and spread more rapidly than others.

A family history of bladder cancer is also a risk factor for the disease. Many cancer experts assert that some people appear to inherit reduced ability to break down certain chemicals, which makes them more sensitive to the cancer-causing effects of tobacco smoke and certain industrial chemicals.

B. Traditional Diagnosis

The gold standard of diagnosing bladder cancer is urine cytology and transurethral (through the urethra) cystoscopy. Urine cytology can be obtained in voided urine or at the time of the cystoscopy (“bladder washing”). Cytology is very specific (a positive result is highly indicative of bladder cancer) but suffers from low sensitivity (a negative result does not exclude the diagnosis of cancer). There are newer urine bound markers for the diagnosis of bladder cancer. These markers are more sensitive but not as specific as urine cytology. They are much more expensive as well. Many patients with a history, signs, and symptoms suspicious for bladder cancer are referred to a urologist or other physician trained in cystoscopy, a procedure in which a flexible tube bearing a camera and various instruments is introduced into the bladder through the urethra. Suspicious lesions may be biopsied and sent for pathologic analysis.

Ninety percent of bladder cancer are transitional cell carcinomas (TCC) that arise from the inner lining of the bladder called the urothelium. The other 10% of tumours are squamous cell carcinoma, adenocarcinoma, sarcoma, small cell carcinoma and secondary deposits from cancers elsewhere in the body.

TCCs are often multifocal, with 30-40% of patients having a more than one tumour at diagnosis. The pattern of growth of TCCs can be papillary, sessile (flat) or carcinoma-in-situ (CIS). The 1973 WHO grading system for TCCs (papilloma, G1, G2 or G3) is most commonly used despite being superseded by the 2004 WHO grading (papillary neoplasm of low malignant potential (PNLMP), low grade and high grade papillary carcinoma. CIS invariably consists of cytologically high grade tumour cells.

Bladder TCC is staged according to the 1997 TNM system:
Ta—non-invasive papillary tumor
T1—invasive but not as far as the muscular bladder layer
T2—invasive into the muscular layer
T3—invasive beyond the muscle into the fat outside the bladder
T4—invasive into surrounding structures like the prostate, uterus or pelvic wall
   The following stages are used to classify the location, size, and spread of the cancer, according to the TNM (tumor, lymph node, and metastases) staging system:
Stage 0: Cancer cells are found only on the inner lining of the bladder.
Stage I: Cancer cells have proliferated to the layer beyond the inner lining of the urinary bladder but not to the muscles of the urinary bladder.
Stage II: Cancer cells have proliferated to the muscles in the bladder wall but not to the fatty tissue that surrounds the urinary bladder.
Stage III: Cancer cells have proliferated to the fatty tissue surrounding the urinary bladder and to the prostate gland, vagina, or uterus, but not to the lymph nodes or other organs.
Stage IV: Cancer cells have proliferated to the lymph nodes, pelvic or abdominal wall, and/or other organs.
Recurrent: Cancer has recurred in the urinary bladder or in another nearby organ after having been treated.

C. Treatment

The treatment of bladder cancer depends on how deep the tumor invades into the bladder wall. Superficial tumors (those not entering the muscle layer) can be “shaved off” using an electrocautery device attached to a cystoscope.

Immunotherapy in the form of BCG instillation is also used to treat and prevent the recurrence of superficial tumors. BCG immunotherapy is effective in up to ⅔ of the cases at this stage. Installations of chemotherapy into the bladder can also be used to treat superficial disease. Bacillus Calmette-Guerin (BCG) has been in use since the 1980's, and is the most proven and effective form of immunotherapy at this point in time. BCG is an inactivated form of the bacterium Mycobacterium tuberculosis, which is given both intravesically mixed in a saline solution and instilled directly into the bladder via a catheter, as well as in the form of a percutaneous vaccine. Although it is not yet totally understood why BCG and other immunotherapies work against cancer, they are thought to elicit an immune response.

It has been shown that BCG induces a variety of cytokines into the urine of patients with superficial TCC, and that some cytokines have antiangiogenic activity. One study demonstrated that interferon-inducible protein 10 (IP-10) and its inducing anti-angiogenic cytokines, interferon-γ and interleukin-12, are increased during intravesical BCG immunotherapy of bladder TCC. These data suggest that, in addition to a cellular immune response, BCG may induce a cytokine-mediated antiangiogenic environment that aids in inhibiting future tumor growth and progression.

Though side effects vary with the individual, the great majority of people find BCG treatments tolerable with side effects being temporary, and some have no adverse reactions at all. Dysuria (pain or difficulty upon urination) and urinary frequency are expected as a consequence of the inflammatory response, and cystitis is the most frequent adverse reaction-occurring in up to 90% of cases. Blood in the urine may occur with cystitis and is seen in one-third of patients. Irritative bladder symptoms are unlikely in the week after the first intravesical BCG. Side effects of BCG are cumulatory, and generally increase with successive treatments. Some people complain of flu like symptoms including fatigue, joint pain and muscle ache.

Untreated, superficial tumors may gradually begin to infiltrate the muscular wall of the bladder. Tumors that infiltrate the bladder require more radical surgery where part or all of the bladder is removed (a cystectomy) and the urinary stream is diverted. In some cases, skilled surgeons can create a substitute bladder (a neobladder) from a segment of intestinal tissue, but this largely depends upon patient preference, age of patient, renal function, and the site of the disease.

A combination of radiation and chemotherapy can also be used to treat invasive disease. It has not yet been determined how the effectiveness of this form of treatment compares to that of radical ablative surgery. There is weak observational evidence from one small study to suggest that the concurrent use of statins is associated with failure of BCG immunotherapy.

### II. FOX PROTEINS

A. FoxA1

FoxA1 is a member of the forkhead class of DNA-binding proteins. These hepatocyte nuclear factors are transcriptional activators for liver-specific transcripts such as albumin and transthyretin, and they also interact with chromatin. Similar family members in mice have roles in the regulation of metabolism and in the differentiation of the pancreas and liver. More specifically, FoxA1 is a transcription factor that is involved in embryonic development, establishment of tissue-specific gene expression and regulation of gene expression in differentiated tissues.

It is thought to act as a ‘pioneer’ factor opening the compacted chromatin for other proteins through interactions with nucleosomal core histones, thereby replacing linker histones at target enhancer and/or promoter sites. It binds DNA with the following consensus sequence:

5′-[AC]A[AT]T[AG]TT[GT][AG][CT]T[CT]-3′

It is proposed to play a role in translating the epigenetic signatures into cell type-specific enhancer-driven transcriptional programs.

Its differential recruitment to chromatin is dependent on distribution of histone H3 methylated at ‘Lys-5’ (H3K4me2) in estrogen-regulated genes. It is involved in the development of multiple endoderm-derived organ systems such as liver, pancreas, lung and prostate; FoxA1 and FoxA2 seem to have at least in part redundant roles

FoxA1 also modulates the transcriptional activity of nuclear hormone receptors. It is involved in ESR1-mediated transcription and is required for ESR1 binding to the NKX2-1 promoter in breast cancer cells. It binds to the RPRM promoter and is required for the estrogen-induced repression of RPRM and is involved in regulation of apoptosis by inhibiting the expression of BCL2. It also is involved in cell cycle regulation by activating expression of CDKN1B, alone or in conjunction with BRCA1.

FoxA1 was originally described as a transcription activator for a number of liver genes such as AFP, albumin, tyrosine aminotransferase, PEPCK, etc. It interacts with the cis-acting regulatory regions of these genes and is involved in glucose homeostasis.

B. FoxA2

FoxA2, like FoxA1, is a member of the forkhead class of DNA-binding proteins. FoxA2 has been linked to sporadic cases of maturity-onset diabetes of the young. Transcript variants encoding different isoforms have been identified for this gene. Also like FoxA1, FoxA2 is a transcription factor that is involved in embryonic development, establishment of tissue-specific gene expression and regulation of gene expression in differentiated tissues and is thought to act as a ‘pioneer’ factor opening the compacted chromatin for other proteins through interactions with nucleosomal core histones and thereby replacing linker histones at target enhancer and/or promoter sites. It binds DNA with the following consensus sequence:

5′-[AC]A[AT]T[AG]TT[GT][AG][CT]T[CT]-3′

In embryonic development, it is required for notochord formation. It is involved in the development of multiple endoderm-derived organ systems such as the liver, pancreas and lungs;

FoxA1 and FoxA2 seem to have at least in part redundant roles. Originally described as a transcription activator for a number of liver genes such as AFP, albumin, tyrosine aminotransferase, PEPCK, etc., FoxA2 interacts with the cis-acting regulatory regions of these genes.

FoxA2 is involved in glucose homeostasis; regulates the expression of genes important for glucose sensing in pancreatic beta-cells and glucose homeostasis. Involved in regulation of fat metabolism. It binds to fibrinogen β promoter and is involved in IL6-induced fibrinogen β transcriptional activation.

### III. PROGNOSTIC DETERMINATIONS IN BLADDER CANCER

In addition to the bladder cancer classification methods described above, the present invention also provides for making predictions on the clinical prospects of a bladder cancer patient. Using information derived from FoxA1 analysis, one can predict whether a bladder cancer patient will be a long term survivor based on a higher levels of FoxA1 expression, with a poor prognosis associated with lower levels. Conversely, information derived from FoxA2 analysis, one can predict whether a bladder cancer patient will be a long term survivor based on a lower levels of FoxA1 expression, with a poor prognosis associated with high levels.

### IV. PROTEIN-BASED DETECTION—IMMUNODETECTION

Thus, in accordance with the present invention, methods are provided for the assaying of FoxA1 and/or FoxA2 in patients suffering from or potentially having bladder cancer. As discussed above, the principle applications of this assay are to: (a) determine what stage of bladder cancer a given patient suffers from; and (b) determine the likelihood and extent of patient survival. In each of these assays, the expression of FoxA1 will be measured.

There are a variety of methods that can be used to assess protein expression. One such approach is to perform protein identification with the use of antibodies. As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting. The term “antibody” also refers to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies, both polyclonal and monoclonal, are also well known in the art (See, e.g., Antibodies: A Laboratory Manual, Cold Spring Harbor Laboratory, 1988; incorporated herein by reference).

In accordance with the present invention, immunodetection methods are provided. Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. The steps of various useful immunodetection methods have been described in the scientific literature, such as, e.g., Doolittle & Ben-Zeev O, 1999; Gulbis & Galand, 1993; De Jager et al., 1993; and Nakamura et al., 1987, each incorporated herein by reference.

In general, the immunobinding methods include obtaining a sample suspected of containing a relevant polypeptide, and contacting the sample with a first antibody under conditions effective to allow the formation of immunocomplexes. In terms of antigen detection, the biological sample analyzed may be any sample that is suspected of containing an antigen, such as, for example, a tissue section or specimen, a homogenized tissue extract, a cell, or even a biological fluid.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any antigens present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. Patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection designed by Charles Cantor uses two different antibodies. A first step biotinylated, monoclonal or polyclonal antibody is used to detect the target antigen(s), and a second step antibody is then used to detect the biotin attached to the complexed biotin. In that method the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

As detailed above, immunoassays are in essence binding assays. Certain immunoassays are the various types of enzyme linked immunosorbent assays (ELISAs) and radioimmunoassays (RIA) known in the art. However, it will be readily appreciated that detection is not limited to such techniques, and Western blotting, dot blotting, FACS analyses, and the like may also be used.

In one exemplary ELISA, the antibodies of the invention are immobilized onto a selected surface exhibiting protein affinity, such as a well in a polystyrene microtiter plate. Then, a test composition suspected of containing the antigen, such as a clinical sample, is added to the wells. After binding and washing to remove non-specifically bound immune complexes, the bound antigen may be detected. Detection is generally achieved by the addition of another antibody that is linked to a detectable label. This type of ELISA is a simple “sandwich ELISA.” Detection may also be achieved by the addition of a second antibody, followed by the addition of a third antibody that has binding affinity for the second antibody, with the third antibody being linked to a detectable label.

In another exemplary ELISA, the samples suspected of containing the antigen are immobilized onto the well surface and then contacted with the anti-ORF message and anti-ORF translated product antibodies of the invention. After binding and washing to remove non-specifically bound immune complexes, the bound anti-ORF message and anti-ORF translated product antibodies are detected. Where the initial anti-ORF message and anti-ORF translated product antibodies are linked to a detectable label, the immune complexes may be detected directly. Again, the immune complexes may be detected using a second antibody that has binding affinity for the first anti-ORF message and anti-ORF translated product antibody, with the second antibody being linked to a detectable label.

Another ELISA in which the antigens are immobilized, involves the use of antibody competition in the detection. In this ELISA, labeled antibodies against an antigen are added to the wells, allowed to bind, and detected by means of their label. The amount of an antigen in an unknown sample is then determined by mixing the sample with the labeled antibodies against the antigen during incubation with coated wells. The presence of an antigen in the sample acts to reduce the amount of antibody against the antigen available for binding to the well and thus reduces the ultimate signal. This is also appropriate for detecting antibodies against an antigen in an unknown sample, where the unlabeled antibodies bind to the antigen-coated wells and also reduces the amount of antigen available to bind the labeled antibodies.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background. The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

The antibodies of the present invention may also be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors, and/or is well known to those of skill in the art (Brown et al., 1990; Abbondanzo et al., 1999; Allred et al., 1990).

Also contemplated in the present invention is the use of immunohistochemistry. This approach uses antibodies to detect and quantify antigens in intact tissue samples. Generally, frozen-sections are prepared by rehydrating frozen “pulverized” tissue at room temperature in phosphate buffered saline (PBS) in small plastic capsules; pelleting the particles by centrifugation; resuspending them in a viscous embedding medium (OCT); inverting the capsule and pelleting again by centrifugation; snap-freezing in −70° C. isopentane; cutting the plastic capsule and removing the frozen cylinder of tissue; securing the tissue cylinder on a cryostat microtome chuck; and cutting 25-50 serial sections.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and cutting up to 50 serial permanent sections.

In still further embodiments, the present invention concerns immunodetection kits for use with the immunodetection methods described above. The kits will include antibodies to FoxA1, and may contain other reagents as well. The immunodetection kits will thus comprise, in suitable container means, a first antibody that binds to FoxA1, and optionally a second and distinct antibody to FoxA1, and optionally one or two antibodies to FoxA2.

In certain embodiments, the antibody to FoxA1 and/or FoxA2 may be pre-bound to a solid support, such as a column matrix, a microtitre plate, a filter, a membrane, a bead or a dipstick. The immunodetection reagents of the kit may take any one of a variety of forms, including antibodies to FoxA1 and/or FoxA2 containing detectable labels. As noted above, a number of exemplary labels are known in the art and all such labels may be employed in connection with the present invention.

The kits may further comprise a suitably aliquoted composition of FoxA1 and/or FoxA2, whether labeled or unlabeled, as may be used to prepare a standard curve for a detection assay. The components of the kits may be packaged either in aqueous media or in lyophilized form. Alternatively or in addition, the kit may comprise a normal cell that does not express FoxA1 and/or FoxA2, and/or a cancer cell that does express FoxA1 and/or FoxA2.

The container means of the kits will generally include at least one vial, test tube, flask, bottle, syringe or other container means, into which the antibody may be placed, or preferably, suitably aliquoted. The kits of the present invention will also typically include a means for containing the antibody, antigen, and any other reagent containers in close confinement for commercial sale. Such containers may include injection or blow-molded plastic containers into which the desired vials are retained.

Type II Assay.

In Type II assay formats, a limited amount of antibody is used (insufficient to bind the entire antigen) a prefixed amount of labeled antigen competes with the unlabeled antigen in test sample for a limited number of antibody binding sites. The concentration of unlabeled antigen in specimen can be determined from the portion of labeled antigen that is bound to the antibody. Since most analyte molecules are not enough large to provide two different epitopes in this method, the response will be inversely proportional to the concentration of antigen in the unknown.

Homogenous and Heterogenous Assay.

The use off either competitive or immunometric assays requires differentiation of bound from free label. This can be archived either by separating bound from free label using a means of removing antibody (heterogeneous) or modulation of signal of the label when antigen is bound to antibody compared to when it is free (homogeneous).

Most solid phase immunoassays belong to the Heterogeneous Assay category. There are many ways of separating bound from free label such as precipitation of antibody, chromatographic method, and solid phase coupling antibody. Homogeneous assays do not require any of separation step to distinguish antigen bound antibody from free antibody. It has an advantage in automation, and typically is faster, easier to perform, and more cost-effective, but its specificity and sensitivity are lower.

Immunochromatography.

There is two different immunochromatography assays based on porous materials—nitrocellulose or nylon membrane. Depending on the liquid migration method, these are classified as lateral flow assay (LFA) or flow through assay (FTA). LFA methods are described in U.S. Pat. No. 6,485,982 is original patent belong to IMA.

2D-Gel Electrophoresis.

2-D electrophoresis begins with 1-D electrophoresis but then separates the molecules by a second property in a direction 90 degrees from the first. In 1-D electrophoresis, proteins (or other molecules) are separated in one dimension, so that all the proteins/molecules will lie along a lane but that the molecules are spread out across a 2-D gel. Because it is unlikely that two molecules will be similar in two distinct properties, so molecules are more effectively separated in 2-D electrophoresis than in 1-D electrophoresis.

The two dimensions that proteins are separated into using this technique can be isoelectric point, protein complex mass in the native state, and protein mass. To separate the proteins by isoelectric point is called isoelectric focusing (IEF). Thereby, a gradient of pH is applied to a gel and an electric potential is applied across the gel, making one end more positive than the other. At all pHs other than their isoelectric point, proteins will be charged. If they are positively charged, they will be pulled towards the more negative end of the gel and if they are negatively charged they will be pulled to the more positive end of the gel. The proteins applied in the first dimension will move along the gel and will accumulate at their isoelectric point; that is, the point at which the overall charge on the protein is 0 (a neutral charge).

A typical second dimensional separation is SDS-PAGE. Before separating the proteins by mass, they are treated with sodium dodecyl sulfate (SDS) along with other reagents (SDS-PAGE in 1-D). This denatures the proteins (that is, it unfolds them into long, straight molecules) and binds a number of SDS molecules roughly proportional to the protein's length. Because a protein's length (when unfolded) is roughly proportional to its mass, this is equivalent to saying that it attaches a number of SDS molecules roughly proportional to the protein's mass. Since the SDS molecules are negatively charged, the result of this is that all of the proteins will have approximately the same mass-to-charge ratio as each other. In addition, proteins will not migrate when they have no charge (a result of the isoelectric focusing step) therefore the coating of the protein in SDS (negatively charged) allows migration of the proteins in the second dimension (NB SDS is not compatible for use in the first dimension as it is charged and a nonionic or zwitterionic detergent needs to be used). In the second dimension, an electric potential is again applied, but at a 90 degree angle from the first field. The proteins will be attracted to the more positive side of the gel proportionally to their mass-to-charge ratio. As previously explained, this ratio will be nearly the same for all proteins. The proteins' progress will be slowed by frictional forces. The gel therefore acts like a molecular sieve when the current is applied, separating the proteins on the basis of their molecular weight with larger proteins being retained higher in the gel and smaller proteins being able to pass through the sieve and reach lower regions of the gel.

Proteins can then be detected by a variety of means, but the most commonly used stains are silver and Coomassie Brilliant Blue staining. In this case, a silver colloid is applied to the gel. The silver binds to cysteine groups within the protein. The silver is darkened by exposure to ultra-violet light. The darkness of the silver can be related to the amount of silver and therefore the amount of protein at a given location on the gel. This measurement can only give approximate amounts, but is adequate for most purposes.

C. Dipstick Technology

U.S. Pat. No. 4,366,241, and Zuk, EP-A 0 143 574 describe migration type assays in which a membrane is impregnated with the reagents needed to perform the assay. An analyte detection zone is provided in which labeled analyte is bound and assay indicia is read.

U.S. Pat. No. 4,770,853, WO 88/08534, and EP-A 0 299 428 describe migration assay devices which incorporate within them reagents which have been attached to colored direct labels, thereby permitting visible detection of the assay results without addition of further substances.

U.S. Pat. No. 4,632,901, disclose a flow-through type immunoassay device comprising antibody (specific to a target antigen analyte) bound to a porous membrane or filter to which is added a liquid sample. As the liquid flows through the membrane, target analyte binds to the antibody. The addition of sample is followed by addition of labeled antibody. The visual detection of labeled antibody provides an indication of the presence of target antigen analyte in the sample.

EP-A 0 125 118, disclose a sandwich type dipstick immunoassay in which immunochemical components such as antibodies are bound to a solid phase. The assay device is “dipped” for incubation into a sample suspected of containing unknown antigen analyte. Enzyme-labeled antibody is then added, either simultaneously or after an incubation period. The device next is washed and then inserted into a second solution containing a substrate for the enzyme. The enzyme-label, if present, interacts with the substrate, causing the formation of colored products which either deposit as a precipitate onto the solid phase or produce a visible color change in the substrate solution.

EP-A 0 282 192, disclose a dipstick device for use in competition type assays.

U.S. Pat. No. 4,313,734 describes the use of gold sol particles as a direct label in a dipstick device.

U.S. Pat. No. 4,786,589 describes a dipstick immunoassay device in which the antibodies have been labeled with formazan.

U.S. Pat. No. 5,656,448 pertains to dipstick immunoassay devices comprising a base member and a single, combined sample contact zone and test zone, wherein the test zone incorporates the use of symbols to detect analytes in a sample of biological fluid. A first immunological component, an anti-immunoglobulin capable of binding to an enzyme-labeled antibody, is immobilized in a control indicator portion. A second immunological component, capable of specifically binding to a target analyte which is bound to the enzyme-labeled antibody to form a sandwich complex, is immobilized in a test indicia portion. The enzyme-labeled antibody produces a visual color differential between a control indicia portion and a non-indicia portion in the test zone upon contact with a substrate. The device additionally includes a first polyol and a color differential enhancing component selected from the group consisting of an inhibitor to the enzyme and a competitive secondary substrate for the enzyme distributed throughout the non-indicia portion of the test zone.

Tissue Histology.

Antibodies to FoxA1 and/or FoxA2 may be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors, and is well known to those of skill in the art (Brown et al., 1990; Abbondanzo et al., 1990; Allred et al., 1990).

Briefly, frozen-sections may be prepared by rehydrating 50 ng of frozen “pulverized” tissue at room temperature in phosphate buffered saline (PBS) in small plastic capsules; pelleting the particles by centrifugation; resuspending them in a viscous embedding medium (OCT); inverting the capsule and/or pelleting again by centrifugation; snap-freezing in −70° C. isopentane; cutting the plastic capsule and/or removing the frozen cylinder of tissue; securing the tissue cylinder on a cryostat microtome chuck; and/or cutting 25-50 serial sections from the capsule. Alternatively, whole frozen tissue samples may be used for serial section cuttings.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections. Again, whole tissue samples may be substituted.

### V. PROTEIN-BASED DETECTION

### Mass Spectrometry

By exploiting the intrinsic properties of mass and charge, mass spectrometry (MS) can resolved and confidently identified a wide variety of complex compounds, including proteins. Traditional quantitative MS has used electrospray ionization (ESI) followed by tandem MS (MS/MS) (Chen et al., 2001; Zhong et al., 2001; Wu et al., 2000) while newer quantitative methods are being developed using matrix assisted laser desorption/ionization (MALDI) followed by time of flight (TOF) MS (Bucknall et al., 2002; Mirgorodskaya et al., 2000; Gobom et al., 2000). In accordance with the present invention, one can generate mass spectrometry profiles that are useful for staging bladder cancers and predicting bladder cancer patient survival by examining FoxA1 expression, and optionally FoxA2 expression.

1. ESI

ESI is a convenient ionization technique developed by Fenn and colleagues (Fenn et al., 1989) that is used to produce gaseous ions from highly polar, mostly nonvolatile biomolecules, including lipids. The sample is injected as a liquid at low flow rates (1-10 μL/min) through a capillary tube to which a strong electric field is applied. The field generates additional charges to the liquid at the end of the capillary and produces a fine spray of highly charged droplets that are electrostatically attracted to the mass spectrometer inlet. The evaporation of the solvent from the surface of a droplet as it travels through the desolvation chamber increases its charge density substantially. When this increase exceeds the Rayleigh stability limit, ions are ejected and ready for MS analysis.

A typical conventional ESI source consists of a metal capillary of typically 0.1-0.3 mm in diameter, with a tip held approximately 0.5 to 5 cm (but more usually 1 to 3 cm) away from an electrically grounded circular interface having at its center the sampling orifice, such as described by Kabarle et al. (1993). A potential difference of between 1 to 5 kV (but more typically 2 to 3 kV) is applied to the capillary by power supply to generate a high electrostatic field (10⁶ to 10⁷ V/m) at the capillary tip. A sample liquid carrying the analyte to be analyzed by the mass spectrometer, is delivered to tip through an internal passage from a suitable source (such as from a chromatograph or directly from a sample solution via a liquid flow controller). By applying pressure to the sample in the capillary, the liquid leaves the capillary tip as a small highly electrically charged droplets and further undergoes desolvation and breakdown to form single or multicharged gas phase ions in the form of an ion beam. The ions are then collected by the grounded (or negatively charged) interface plate and led through an the orifice into an analyzer of the mass spectrometer. During this operation, the voltage applied to the capillary is held constant. Aspects of construction of ESI sources are described, for example, in U.S. Pat. Nos. 5,838,002; 5,788,166; 5,757,994; RE 35,413; and 5,986,258.

2. ESI/MS/MS

In ESI tandem mass spectroscopy (ESI/MS/MS), one is able to simultaneously analyze both precursor ions and product ions, thereby monitoring a single precursor product reaction and producing (through selective reaction monitoring (SRM)) a signal only when the desired precursor ion is present. When the internal standard is a stable isotope-labeled version of the analyte, this is known as quantification by the stable isotope dilution method. This approach has been used to accurately measure pharmaceuticals (Zweigenbaum et al., 2000; Zweigenbaum et al., 1999) and bioactive peptides (Desiderio et al., 1996; Lovelace et al., 1991). Newer methods are performed on widely available MALDI-TOF instruments, which can resolve a wider mass range and have been used to quantify metabolites, peptides, and proteins. Larger molecules such as peptides can be quantified using unlabeled homologous peptides as long as their chemistry is similar to the analyte peptide (Duncan et al., 1993; Bucknall et al., 2002). Protein quantification has been achieved by quantifying tryptic peptides (Mirgorodskaya et al., 2000). Complex mixtures such as crude extracts can be analyzed, but in some instances sample clean up is required (Nelson et al., 1994; Gobom et al., 2000).

3. SIMS

Secondary ion mass spectroscopy, or SIMS, is an analytical method that uses ionized particles emitted from a surface for mass spectroscopy at a sensitivity of detection of a few parts per billion. The sample surface is bombarded by primary energetic particles, such as electrons, ions (e.g., O, Cs), neutrals or even photons, forcing atomic and molecular particles to be ejected from the surface, a process called sputtering. Since some of these sputtered particles carry a charge, a mass spectrometer can be used to measure their mass and charge. Continued sputtering permits measuring of the exposed elements as material is removed. This in turn permits one to construct elemental depth profiles. Although the majority of secondary ionized particles are electrons, it is the secondary ions which are detected and analysis by the mass spectrometer in this method.

4. LD-MS and LDLPMS

Laser desorption mass spectroscopy (LD-MS) involves the use of a pulsed laser, which induces desorption of sample material from a sample site—effectively, this means vaporization of sample off of the sample substrate. This method is usually only used in conjunction with a mass spectrometer, and can be performed simultaneously with ionization if one uses the right laser radiation wavelength.

When coupled with Time-of-Flight (TOF) measurement, LD-MS is referred to as LDLPMS (Laser Desorption Laser Photoionization Mass Spectroscopy). The LDLPMS method of analysis gives instantaneous volatilization of the sample, and this form of sample fragmentation permits rapid analysis without any wet extraction chemistry. The LDLPMS instrumentation provides a profile of the species present while the retention time is low and the sample size is small. In LDLPMS, an impactor strip is loaded into a vacuum chamber. The pulsed laser is fired upon a certain spot of the sample site, and species present are desorbed and ionized by the laser radiation. This ionization also causes the molecules to break up into smaller fragment-ions. The positive or negative ions made are then accelerated into the flight tube, being detected at the end by a microchannel plate detector. Signal intensity, or peak height, is measured as a function of travel time. The applied voltage and charge of the particular ion determines the kinetic energy, and separation of fragments are due to different size causing different velocity. Each ion mass will thus have a different flight-time to the detector.

One can either form positive ions or negative ions for analysis. Positive ions are made from regular direct photoionization, but negative ion formation require a higher powered laser and a secondary process to gain electrons. Most of the molecules that come off the sample site are neutrals, and thus can attract electrons based on their electron affinity. The negative ion formation process is less efficient than forming just positive ions. The sample constituents will also affect the outlook of a negative ion spectra.

Other advantages with the LDLPMS method include the possibility of constructing the system to give a quiet baseline of the spectra because one can prevent coevolved neutrals from entering the flight tube by operating the instrument in a linear mode. Also, in environmental analysis, the salts in the air and as deposits will not interfere with the laser desorption and ionization. This instrumentation also is very sensitive, known to detect trace levels in natural samples without any prior extraction preparations.

5. MALDI-TOF-MS

Since its inception and commercial availability, the versatility of MALDI-TOF-MS has been demonstrated convincingly by its extensive use for qualitative analysis. For example, MALDI-TOF-MS has been employed for the characterization of synthetic polymers (Marie et al., 2000; Wu et al., 1998). peptide and protein analysis (Roepstorff et al., 2000; Nguyen et al., 1995), DNA and oligonucleotide sequencing (Miketova et al., 1997; Faulstich et al., 1997; Bentzley et al., 1996), and the characterization of recombinant proteins (Kanazawa et al., 1999; Villanueva et al., 1999). Recently, applications of MALDI-TOF-MS have been extended to include the direct analysis of biological tissues and single cell organisms with the aim of characterizing endogenous peptide and protein constituents (Li et al., 2000; Lynn et al., 1999; Stoeckli et al., 2001; Caprioli et al., 1997; Chaurand et al., 1999; Jespersen et al., 1999).

The properties that make MALDI-TOF-MS a popular qualitative tool—its ability to analyze molecules across an extensive mass range, high sensitivity, minimal sample preparation and rapid analysis times—also make it a potentially useful quantitative tool. MALDI-TOF-MS also enables non-volatile and thermally labile molecules to be analyzed with relative ease. It is therefore prudent to explore the potential of MALDI-TOF-MS for quantitative analysis in clinical settings, for toxicological screenings, as well as for environmental analysis. In addition, the application of MALDI-TOF-MS to the quantification of peptides and proteins is particularly relevant. The ability to quantify intact proteins in biological tissue and fluids presents a particular challenge in the expanding area of proteomics and investigators urgently require methods to accurately measure the absolute quantity of proteins. While there have been reports of quantitative MALDI-TOF-MS applications, there are many problems inherent to the MALDI ionization process that have restricted its widespread use (Kazmaier et al., 1998; Horak et al., 2001; Gobom et al., 2000; Wang et al., 2000; Desiderio et al., 2000). These limitations primarily stem from factors such as the sample/matrix heterogeneity, which are believed to contribute to the large variability in observed signal intensities for analytes, the limited dynamic range due to detector saturation, and difficulties associated with coupling MALDI-TOF-MS to on-line separation techniques such as liquid chromatography. Combined, these factors are thought to compromise the accuracy, precision, and utility with which quantitative determinations can be made.

Because of these difficulties, practical examples of quantitative applications of MALDI-TOF-MS have been limited. Most of the studies to date have focused on the quantification of low mass analytes, in particular, alkaloids or active ingredients in agricultural or food products (Wang et al., 1999; Jiang et al., 2000; Wang et al., 2000; Yang et al., 2000; Wittmann et al., 2001), whereas other studies have demonstrated the potential of MALDI-TOF-MS for the quantification of biologically relevant analytes such as neuropeptides, proteins, antibiotics, or various metabolites in biological tissue or fluid (Muddiman et al., 1996; Nelson et al., 1994; Duncan et al., 1993; Gobom et al., 2000; Wu et al., 1997; Mirgorodskaya et al., 2000). In earlier work it was shown that linear calibration curves could be generated by MALDI-TOF-MS provided that an appropriate internal standard was employed (Duncan et al., 1993). This standard can “correct” for both sample-to-sample and shot-to-shot variability. Stable isotope labeled internal standards (isotopomers) give the best result.

With the marked improvement in resolution available on modern commercial instruments, primarily because of delayed extraction (Bahr et al., 1997; Takach et al., 1997), the opportunity to extend quantitative work to other examples is now possible; not only of low mass analytes, but also biopolymers. Of particular interest is the prospect of absolute multi-component quantification in biological samples (e.g., proteomics applications).

The properties of the matrix material used in the MALDI method are critical. Only a select group of compounds is useful for the selective desorption of proteins and polypeptides. A review of all the matrix materials available for peptides and proteins shows that there are certain characteristics the compounds must share to be analytically useful. Despite its importance, very little is known about what makes a matrix material “successful” for MALDI. The few materials that do work well are used heavily by all MALDI practitioners and new molecules are constantly being evaluated as potential matrix candidates. With a few exceptions, most of the matrix materials used are solid organic acids. Liquid matrices have also been investigated, but are not used routinely.

### VI. NUCLEIC ACID DETECTION

In alternative embodiments for detecting protein expression, one may assay for gene transcription. For example, an indirect method for detecting protein expression is to detect mRNA transcripts from which the proteins are made. The following is a discussion of such methods, which are applicable particularly to FoxA1 and/or FoxA2 in the context of the present invention.

1. Hybridization

There are a variety of ways by which one can assess gene expression. These methods either look at protein or at mRNA levels. Methods looking at mRNAs all fundamentally rely, at a basic level, on nucleic acid hybridization. Hybridization is defined as the ability of a nucleic acid to selectively form duplex molecules with complementary stretches of DNAs and/or RNAs. Depending on the application envisioned, one would employ varying conditions of hybridization to achieve varying degrees of selectivity of the probe or primers for the target sequence.

Typically, a probe or primer of between 13 and 100 nucleotides, preferably between 17 and 100 nucleotides in length up to 1-2 kilobases or more in length will allow the formation of a duplex molecule that is both stable and selective. Molecules having complementary sequences over contiguous stretches greater than 20 bases in length are generally preferred, to increase stability and selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications, for example, lower stringency conditions may be used. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples.

In general, it is envisioned that the probes or primers described herein will be useful as reagents in solution hybridization, as in PCR™, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section of the Specification are incorporated herein by reference.

2. Amplification of Nucleic Acids

Since many mRNAs are present in relatively low abundance, nucleic acid amplification greatly enhances the ability to assess expression. The general concept is that nucleic acids can be amplified using paired primers flanking the region of interest. The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids corresponding to selected genes are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids contain one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemiluminescence, radioactive scintigraphy of incorporated radiolabel or fluorescent label or even via a system using electrical and/or thermal impulse signals.

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1988, each of which is incorporated herein by reference in their entirety.

A reverse transcriptase PCR™ amplification procedure may be performed to quantify the amount of mRNA amplified. Methods of reverse transcribing RNA into cDNA are well known (see Sambrook et al., 1989). Alternative methods for reverse transcription utilize thermostable DNA polymerases. These methods are described in WO 90/07641. Polymerase chain reaction methodologies are well known in the art. Representative methods of RT-PCR are described in U.S. Pat. No. 5,882,864.

Whereas standard PCR usually uses one pair of primers to amplify a specific sequence, multiplex-PCR (MPCR) uses multiple pairs of primers to amplify many sequences simultaneously (Chamberlan et al., 1990). The presence of many PCR primers in a single tube could cause many problems, such as the increased formation of misprimed PCR products and “primer dimers”, the amplification discrimination of longer DNA fragment and so on. Normally, MPCR buffers contain a Taq Polymerase additive, which decreases the competition among amplicons and the amplification discrimination of longer DNA fragment during MPCR. MPCR products can further be hybridized with gene-specific probe for verification. Theoretically, one should be able to use as many as primers as necessary. However, due to side effects (primer dimers, misprimed PCR products, etc.) caused during MPCR, there is a limit (less than 20) to the number of primers that can be used in a MPCR reaction. See also European Application No. 0 364 255 and Mueller and Wold (1989).

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ and oligonucleotide ligase assay (OLA), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

Qbeta Replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include transcription-based amplification systems (TAS), including nucleic acid sequence based amplification (NASBA) and 3SR (Kwoh et al., 1989; Gingeras et al., PCT Application WO 88/10315, incorporated herein by reference in their entirety). European Application No. 329 822 disclose a nucleic acid amplification process involving cyclically synthesizing single-stranded RNA (“ssRNA”), ssDNA, and double-stranded DNA (dsDNA), which may be used in accordance with the present invention.

PCT Application WO 89/06700 (incorporated herein by reference in its entirety) disclose a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “race” and “one-sided PCR” (Frohman, 1990; Ohara et al., 1989).

3. Detection of Nucleic Acids

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 1989). Separated amplification products may be cut out and eluted from the gel for further manipulation. Using low melting point agarose gels, the separated band may be removed by heating the gel, followed by extraction of the nucleic acid.

Separation of nucleic acids may also be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art (see Sambrook et al., 1989). One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

4. Nucleic Acid Arrays

Microarrays comprise a plurality of polymeric molecules spatially distributed over, and stably associated with, the surface of a substantially planar substrate, e.g., biochips. Microarrays of polynucleotides have been developed and find use in a variety of applications, such as screening and DNA sequencing. One area in particular in which microarrays find use is in gene expression analysis.

In gene expression analysis with microarrays, an array of “probe” oligonucleotides is contacted with a nucleic acid sample of interest, i.e., target, such as polyA mRNA from a particular tissue type. Contact is carried out under hybridization conditions and unbound nucleic acid is then removed. The resultant pattern of hybridized nucleic acid provides information regarding the genetic profile of the sample tested. Methodologies of gene expression analysis on microarrays are capable of providing both qualitative and quantitative information.

A variety of different arrays which may be used are known in the art. The probe molecules of the arrays which are capable of sequence specific hybridization with target nucleic acid may be polynucleotides or hybridizing analogues or mimetics thereof, including: nucleic acids in which the phosphodiester linkage has been replaced with a substitute linkage, such as phosphorothioate, methylimino, methylphosphonate, phosphoramidate, guanidine and the like; nucleic acids in which the ribose subunit has been substituted, e.g., hexose phosphodiester; peptide nucleic acids; and the like. The length of the probes will generally range from 10 to 1000 nts, where in some embodiments the probes will be oligonucleotides and usually range from 15 to 150 nts and more usually from 15 to 100 nts in length, and in other embodiments the probes will be longer, usually ranging in length from 150 to 1000 nts, where the polynucleotide probes may be single- or double-stranded, usually single-stranded, and may be PCR fragments amplified from cDNA.

The probe molecules on the surface of the substrates will correspond to selected genes being analyzed and be positioned on the array at a known location so that positive hybridization events may be correlated to expression of a particular gene in the physiological source from which the target nucleic acid sample is derived. The substrates with which the probe molecules are stably associated may be fabricated from a variety of materials, including plastics, ceramics, metals, gels, membranes, glasses, and the like. The arrays may be produced according to any convenient methodology, such as preforming the probes and then stably associating them with the surface of the support or growing the probes directly on the support. A number of different array configurations and methods for their production are known to those of skill in the art and disclosed in U.S. Pat. Nos. 5,445,934, 5,532,128, 5,556,752, 5,242,974, 5,384,261, 5,405,783, 5,412,087, 5,424,186, 5,429,807, 5,436,327, 5,472,672, 5,527,681, 5,529,756, 5,545,531, 5,554,501, 5,561,071, 5,571,639, 5,593,839, 5,599,695, 5,624,711, 5,658,734, 5,700,637, and 6,004,755.

Following hybridization, where non-hybridized labeled nucleic acid is capable of emitting a signal during the detection step, a washing step is employed where unhybridized labeled nucleic acid is removed from the support surface, generating a pattern of hybridized nucleic acid on the substrate surface. A variety of wash solutions and protocols for their use are known to those of skill in the art and may be used.

Where the label on the target nucleic acid is not directly detectable, one then contacts the array, now comprising bound target, with the other member(s) of the signal producing system that is being employed. For example, where the label on the target is biotin, one then contacts the array with streptavidin-fluorescer conjugate under conditions sufficient for binding between the specific binding member pairs to occur. Following contact, any unbound members of the signal producing system will then be removed, e.g., by washing. The specific wash conditions employed will necessarily depend on the specific nature of the signal producing system that is employed, and will be known to those of skill in the art familiar with the particular signal producing system employed.

The resultant hybridization pattern(s) of labeled nucleic acids may be visualized or detected in a variety of ways, with the particular manner of detection being chosen based on the particular label of the nucleic acid, where representative detection means include scintillation counting, autoradiography, fluorescence measurement, calorimetric measurement, light emission measurement and the like.

Prior to detection or visualization, where one desires to reduce the potential for a mismatch hybridization event to generate a false positive signal on the pattern, the array of hybridized target/probe complexes may be treated with an endonuclease under conditions sufficient such that the endonuclease degrades single stranded, but not double stranded DNA. A variety of different endonucleases are known and may be used, where such nucleases include: mung bean nuclease, S1 nuclease, and the like. Where such treatment is employed in an assay in which the target nucleic acids are not labeled with a directly detectable label, e.g., in an assay with biotinylated target nucleic acids, the endonuclease treatment will generally be performed prior to contact of the array with the other member(s) of the signal producing system, e.g., fluorescent-streptavidin conjugate. Endonuclease treatment, as described above, ensures that only end-labeled target/probe complexes having a substantially complete hybridization at the 3′ end of the probe are detected in the hybridization pattern.

Following hybridization and any washing step(s) and/or subsequent treatments, as described above, the resultant hybridization pattern is detected. In detecting or visualizing the hybridization pattern, the intensity or signal value of the label will be not only be detected but quantified, by which is meant that the signal from each spot of the hybridization will be measured and compared to a unit value corresponding the signal emitted by known number of end-labeled target nucleic acids to obtain a count or absolute value of the copy number of each end-labeled target that is hybridized to a particular spot on the array in the hybridization pattern.

### VI. GENE THERAPY

In another embodiment, the present invention provides for the administration of a gene therapy vector encoding one or more genes identified as being downregulated in bladder cancers. Alternatively, for genes that are overexpressed in bladder cancers, the transgenes may provide for reduced expression of appropriate targets. Various aspects of gene delivery and expression are set forth below.

1. Therapeutic Transgenes

Thus, in accordance with the present invention, there are provided methods of treating cancer utilizing FoxA1. By increasing the expression of this gene product, therapeutic benefit may be provided to patients.

2. Inhibitory Nucleic Acids

In another embodiment within the present invention, there are provided methods of treating cancer utilizing FoxA2 inhibitors. By decreasing the expression of this gene product, therapeutic benefit may be provided to patients.

i. Antisense

The term “antisense” nucleic acid refers to oligo- and polynucleotides complementary to bases sequences of a target DNA or RNA. When introduced into a cell, antisense molecules hybridize to a target nucleic acid and interfere with its transcription, transport, processing, splicing or translation. Targeting double-stranded DNA leads to triple helix formation; targeting RNA will lead to double-helix formation.

Antisense constructs may be designed to bind to the promoter or other control regions, exons, introns or even exon-intron boundaries of a gene. Antisense RNA constructs, or DNA encoding such antisense RNA's, may be employed to inhibit gene transcription or translation within a host cell. Nucleic acid sequences which comprise “complementary nucleotides” are those which are capable of base-pairing according to the standard Watson-Crick complementarity rules. That is, that the larger purines will base pair with the smaller pyrimidines to form combinations of guanine paired with cytosine (G:C) and adenine paired with either thymine in the case of DNA (A:T), or uracil (A:U) in the case of RNA. Inclusion of less common bases such as inosine, 5-methylcytosine, 6-methyladenine, hypoxanthine and others in hybridizing sequences does not interfere with pairing.

As used herein, the terms “complementary” and “antisense sequences” mean nucleic acid sequences that are substantially complementary over their entire length and have very few base mismatches. For example, nucleic acid sequences of fifteen bases in length may be termed complementary when they have complementary nucleotides at thirteen or fourteen positions. Naturally, nucleic acid sequences with are “completely complementary” will be nucleic acid sequences which have perfect base pair matching with the target sequences, i.e., no mismatches. Other sequences with lower degrees of homology are contemplated. For example, an antisense construct with limited regions of high homology, but overall containing a lower degree (50% or less) total homology, may be used.

While all or part of the gene sequence may be employed in the context of antisense construction, statistically, any sequence of 17 bases long should occur only once in the human genome and, therefore, suffice to specify a unique target. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that oligonucleotides of 8, 9, 10, 11, 12, 13, 14, 15, 16, 17, 18, 19, 20 or more base pairs will be used. One can readily determine whether a given antisense nucleic acid is effective at targeting a gene simply by testing the construct in vitro to determine whether the gene's function or expression is affected.

In certain embodiments, one may wish to employ antisense constructs which include other elements, for example, those which include C-5 propyne pyrimidines. Oligonucleotides which contain C-5 propyne analogs of uridine and cytidine have been shown to bind RNA with high affinity and to be potent inhibitors or gene expression (Wagner et al., 1993).

ii. Single Chain Antibodies

Naturally-occurring antibodies (of isotype IgG) produced by B cells, consist of four polypeptide chains. Two heavy chains (composed of four immunoglobulin domains) and two light chains (made up of two immunoglobulin domains) are held together by disulphide bonds. The bulk of the antibody complex is made up of constant immunoglobulin domains. These have a conserved amino acid sequence, and exhibit low variability. Different classes of constant regions in the stem of the antibody generate different isotypes of antibody with differing properties. The recognition properties of the antibody are carried by the variable regions (VH and VL) at the ends of the arms. Each variable domain contains three hypervariable regions known as complementarity determining regions, or CDRs. The CDRs come together in the final tertiary structure to form an antigen binding pocket. The human genome contains multiple fragments encoding portions of the variable domains in regions of the immunoglobulin gene cluster known as V, D and J. During B cell development these regions undergo recombination to generate a broad diversity of antibody affinities. As these B cell populations mature in the presence of a target antigen, hypermutation of the variable region takes place, with the B cells producing the most active antibodies being selected for further expansion in a process known as affinity maturation.

A major breakthrough was the generation of monoclonal antibodies, pure populations of antibodies with the same affinity. This was achieved by fusing B cells taken from immunized animals with myeloma cells. This generates a population of immortal hybridomas, from which the required clones can be selected. Monoclonal antibodies are very important research tools, and have been used in some therapies. However, they are very expensive and difficult to produce, and if used in a therapeutic context, can elicit and immune response which will destroy the antibody. This can be reduced in part by humanizing the antibody by grafting the CDRs from the parent monoclonal into the backbone of a human IgG antibody. It may be better to deliver antibodies by gene therapy, as this would hopefully provide a constant localized supply of antibody following a single dose of vector. The problems of vector design and delivery are dealt with elsewhere, but antibodies in their native form, consisting of two different polypeptide chains which need to be generated in approximately equal amounts and assembled correctly are not good candidates for gene therapy. However, it is possible to create a single polypeptide which can retain the antigen binding properties of a monoclonal antibody.

The variable regions from the heavy and light chains (VH and VL) are both approximately 110 amino acids long. They can be linked by a 15 amino acid linker (e.g., (glycine₄serine)₃), which has sufficient flexibility to allow the two domains to assemble a functional antigen binding pocket. Addition of various signal sequences allows the scFv to be targeted to different organelles within the cell, or to be secreted. Addition of the light chain constant region (Ck) allows dimerization via disulphide bonds, giving increased stability and avidity. However, there is evidence that scFvs spontaneously multimerize, with the extent of aggregation (presumably via exposed hydrophobic surfaces) being dependent on the length of the glycine-serine linker.

The variable regions for constructing the scFv are obtained as follows. Using a monoclonal antibody against the target of interest, it is a simple procedure to use RT-PCR to clone out the variable regions from mRNA extracted from the parent hybridoma. Degenerate primers targeted to the relatively invariant framework regions can be used. Expression constructs are available with convenient cloning sites for the insertion of the cloned variable regions.

iii. siRNA

RNA interference (also referred to as “RNA-mediated interference” or RNAi) is a mechanism by which gene expression can be reduced or eliminated. Double-stranded RNA (dsRNA) has been observed to mediate the reduction, which is a multi-step process. dsRNA activates post-transcriptional gene expression surveillance mechanisms that appear to function to defend cells from virus infection and transposon activity (Fire et al., 1998; Grishok et al., 2000; Ketting et al., 1999; Lin and Avery et al., 1999; Montgomery et al., 1998; Sharp and Zamore, 2000; Tabara et al., 1999). Activation of these mechanisms targets mature, dsRNA-complementary mRNA for destruction. RNAi offers major experimental advantages for study of gene function. These advantages include a very high specificity, ease of movement across cell membranes, and prolonged down-regulation of the targeted gene (Fire et al., 1998; Grishok et al., 2000; Ketting et al., 1999; Lin and Avery et al., 1999; Montgomery et al., 1998; Sharp et al., 1999; Sharp and Zamore, 2000; Tabara et al., 1999). Moreover, dsRNA has been shown to silence genes in a wide range of systems, including plants, protozoans, fungi, C. elegans, Trypanasoma, Drosophila, and mammals (Grishok et al., 2000; Sharp et al., 1999; Sharp and Zamore, 2000; Elbashir et al., 2001). It is generally accepted that RNAi acts post-transcriptionally, targeting RNA transcripts for degradation. It appears that both nuclear and cytoplasmic RNA can be targeted (Bosher and Labouesse, 2000).

siRNAs must be designed so that they are specific and effective in suppressing the expression of the genes of interest. Methods of selecting the target sequences, i.e., those sequences present in the gene or genes of interest to which the siRNAs will guide the degradative machinery, are directed to avoiding sequences that may interfere with the siRNA's guide function while including sequences that are specific to the gene or genes. Typically, siRNA target sequences of about 21 to 23 nucleotides in length are most effective. This length reflects the lengths of digestion products resulting from the processing of much longer RNAs as described above (Montgomery et al., 1998).

The making of siRNAs has been mainly through direct chemical synthesis; through processing of longer, double-stranded RNAs through exposure to Drosophila embryo lysates; or through an in vitro system derived from S2 cells. Use of cell lysates or in vitro processing may further involve the subsequent isolation of the short, 21-23 nucleotide siRNAs from the lysate, etc., making the process somewhat cumbersome and expensive. Chemical synthesis proceeds by making two single-stranded RNA-oligomers followed by the annealing of the two single-stranded oligomers into a double-stranded RNA. Methods of chemical synthesis are diverse. Non-limiting examples are provided in U.S. Pat. Nos. 5,889,136, 4,415,723, and 4,458,066, expressly incorporated herein by reference, and in Wincott et al. (1995).

Several further modifications to siRNA sequences have been suggested in order to alter their stability or improve their effectiveness. It is suggested that synthetic complementary 21-mer RNAs having di-nucleotide overhangs (i.e., 19 complementary nucleotides+3′ non-complementary dimers) may provide the greatest level of suppression. These protocols primarily use a sequence of two (2′-deoxy) thymidine nucleotides as the di-nucleotide overhangs. These dinucleotide overhangs are often written as dTdT to distinguish them from the typical nucleotides incorporated into RNA. The literature has indicated that the use of dT overhangs is primarily motivated by the need to reduce the cost of the chemically synthesized RNAs. It is also suggested that the dTdT overhangs might be more stable than UU overhangs, though the data available shows only a slight (<20%) improvement of the dTdT overhang compared to an siRNA with a UU overhang.

Chemically synthesized siRNAs are found to work optimally when they are in cell culture at concentrations of 25-100 nM, but concentrations of about 100 nM have achieved effective suppression of expression in mammalian cells. siRNAs have been most effective in mammalian cell culture at about 100 nM. In several instances, however, lower concentrations of chemically synthesized siRNA have been used (Caplen, et al., 2000; Elbashir et al., 2001).

WO 99/32619 and WO 01/68836 suggest that RNA for use in siRNA may be chemically or enzymatically synthesized. Both of these texts are incorporated herein in their entirety by reference. The enzymatic synthesis contemplated in these references is by a cellular RNA polymerase or a bacteriophage RNA polymerase (e.g., T3, T7, SP6) via the use and production of an expression construct as is known in the art. For example, see U.S. Pat. No. 5,795,715. The contemplated constructs provide templates that produce RNAs that contain nucleotide sequences identical to a portion of the target gene. The length of identical sequences provided by these references is at least 25 bases, and may be as many as 400 or more bases in length. An important aspect of this reference is that the authors contemplate digesting longer dsRNAs to 21-25mer lengths with the endogenous nuclease complex that converts long dsRNAs to siRNAs in vivo. They do not describe or present data for synthesizing and using in vitro transcribed 21-25mer dsRNAs. No distinction is made between the expected properties of chemical or enzymatically synthesized dsRNA in its use in RNA interference.

Similarly, WO 00/44914, incorporated herein by reference, suggests that single strands of RNA can be produced enzymatically or by partial/total organic synthesis. Preferably, single-stranded RNA is enzymatically synthesized from the PCR products of a DNA template, preferably a cloned cDNA template and the RNA product is a complete transcript of the cDNA, which may comprise hundreds of nucleotides. WO 01/36646, incorporated herein by reference, places no limitation upon the manner in which the siRNA is synthesized, providing that the RNA may be synthesized in vitro or in vivo, using manual and/or automated procedures. This reference also provides that in vitro synthesis may be chemical or enzymatic, for example using cloned RNA polymerase (e.g., T3, T7, SP6) for transcription of the endogenous DNA (or cDNA) template, or a mixture of both. Again, no distinction in the desirable properties for use in RNA interference is made between chemically or enzymatically synthesized siRNA.

U.S. Pat. No. 5,795,715 reports the simultaneous transcription of two complementary DNA sequence strands in a single reaction mixture, wherein the two transcripts are immediately hybridized. The templates used are preferably of between 40 and 100 base pairs, and which is equipped at each end with a promoter sequence. The templates are preferably attached to a solid surface. After transcription with RNA polymerase, the resulting dsRNA fragments may be used for detecting and/or assaying nucleic acid target sequences.

### VII. PHARMACEUTICAL FORMULATIONS AND ROUTES OF ADMINISTRATION

Where clinical applications are contemplated, it will be necessary to prepare pharmaceutical compositions in a form appropriate for the intended application. Generally, this will entail preparing compositions that are essentially free of pyrogens, as well as other impurities that could be harmful to humans or animals.

The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Supplementary active ingredients also can be incorporated into the compositions.

Administration of these compositions according to the present invention will be via any common route so long as the target tissue is available via that route. This includes intradermal, subcutaneous, intramuscular, intraperitoneal or intravenous injection. In particular, intratumoral routes and sites local and regional to tumors are contemplated. Such compositions would normally be administered as pharmaceutically acceptable compositions, described supra.

The active compounds also may be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy administration by a syringe is possible. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

For oral administration the polypeptides of the present invention may be incorporated with excipients that may include water, binders, abrasives, flavoring agents, foaming agents, and humectants.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

### VIII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

FOXA1 Expression Correlates with Uroplakin Expression In Vitro and in Human Bladder Tissue.

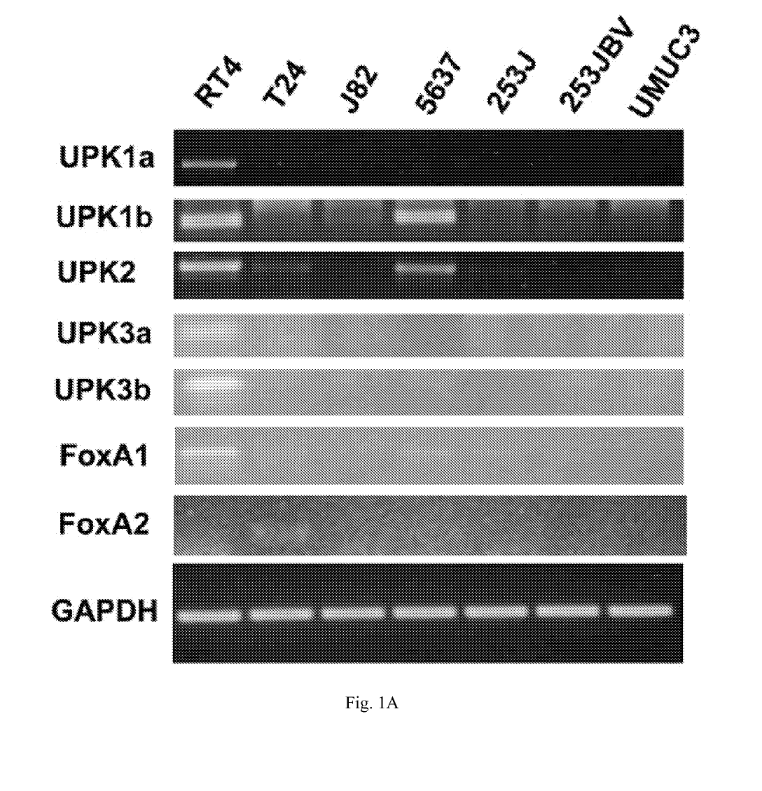

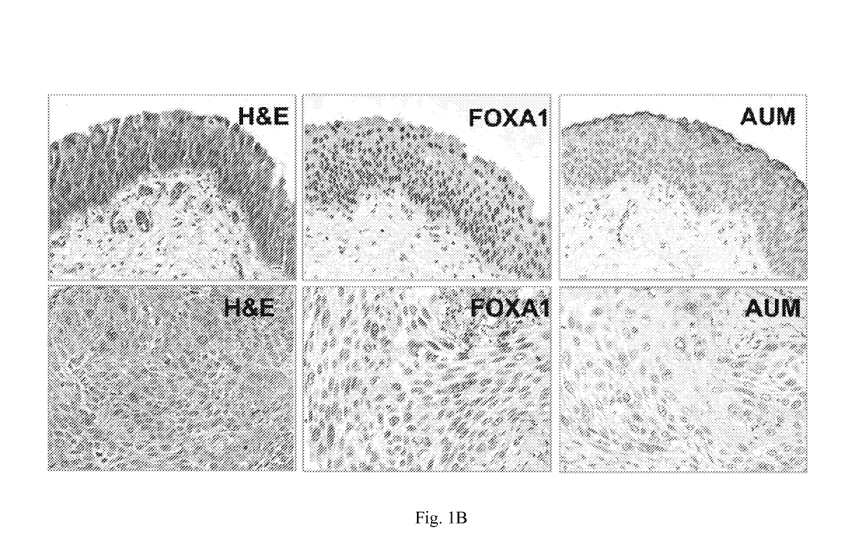

A panel of commonly used urothelial cell lines was screened via traditional RT-PCR for the presence of FOXA1, FOXA2, and FOXA3 transcripts, as well as members of the uroplakin (UPK) family. HepG2 cells, which express each member of the FOXA subfamily, were used as positive controls (data not shown). As can be seen in FIG. 1A, RT4 cells exhibited robust expression of FOXA1 and as previously reported, UPK family members. FOXA2 expression was detected in T24 cells, but was not correlated with UPK family member expression. FOXA3 was not detected in any tested cell line (data not shown). As shown in FIG. 1B, decreased FOXA1 expression is associated with decreased UPK expression in human tissue. Archival normal adjacent tissue (top panel) and bladder tumor was immunostained with a pan-UPK antibody, AUM as well as an antibody directed against FOXA1. These results suggest that decreased FOXA1 is associated with decreased UPK expression in human urothelial carcinoma cell lines and human tumor samples.

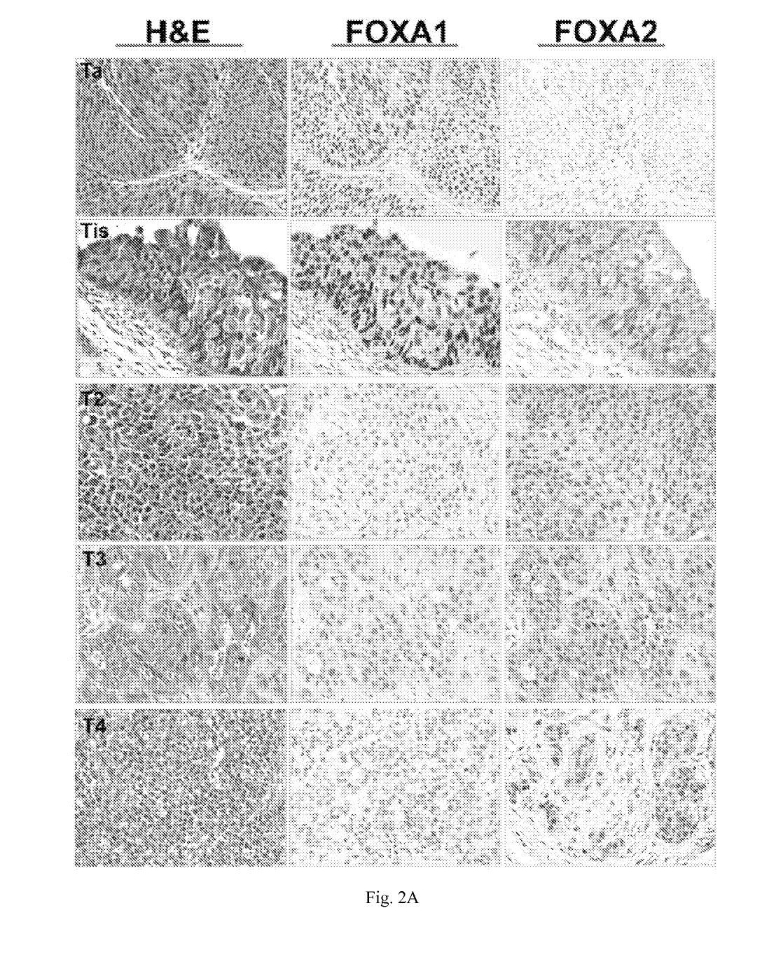

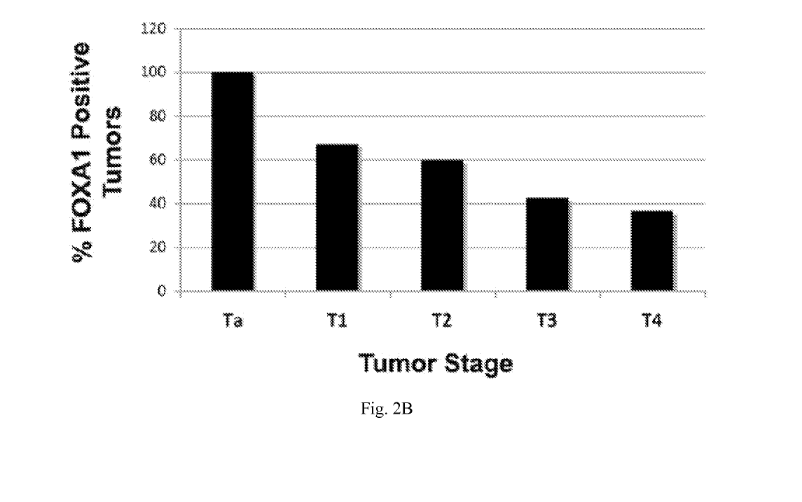

FIG. 2: FOXA1 Expression is Lost in Most High Grade, Advanced Stage Muscle Invasive Bladder Cancers, in Subset of Lymph Node Metastases, and in Keratinizing Squamous Metaplasia of Bladder Urothelium, and is Decreased in Squamous Cell Carcinoma (SCC) of the Urinary Bladder.

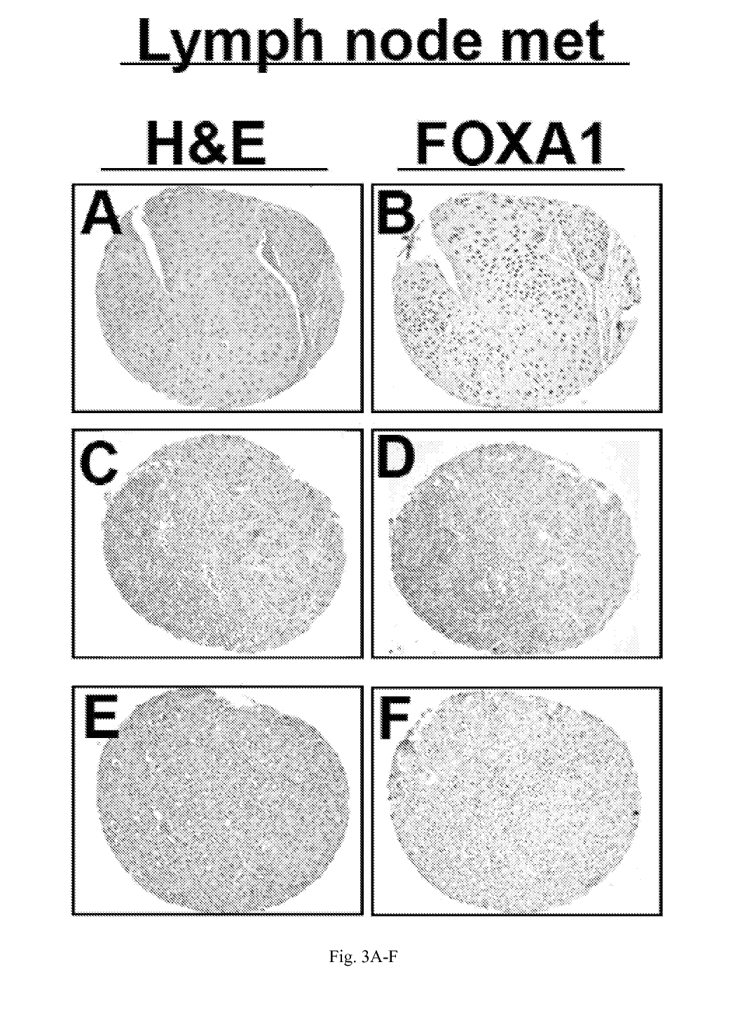

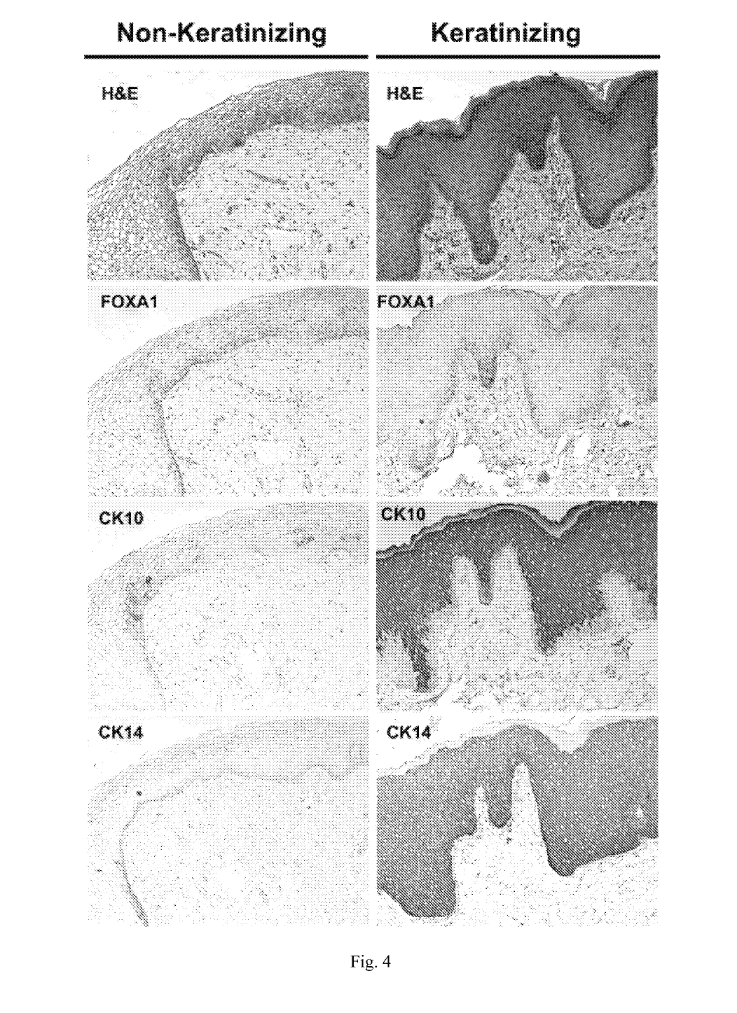

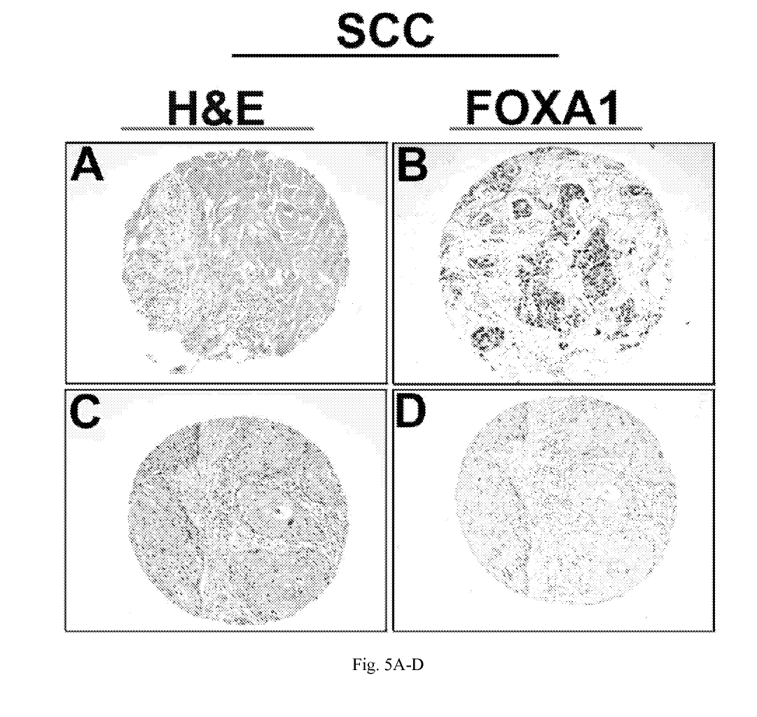

Serial sections of AJCC stage Tis, Ta, T1, T2, T3 and T4 bladder tumors were immunostained for H&E, FOXA1 and FOXA2. Representative cases are illustrated in FIG. 2. As can be seen, loss of FOXA1 staining was observed with advanced tumor stage (p<0.001). A small subset of invasive tumors exhibited nuclear expression of FOXA2. FIGS. 3A, 3C and 3F show H&E standing of FOXA1-positive (FIG. 3B) and FOXA1-negative (FIGS. 3D and 3F) metastatic lymph node samples isolated from bladder cancer patients. Transitional (urothelial) cell carcinoma (FIGS. 3A and 3B) and squamous cell carcinomas (FIGS. 3C-D and 3E-F) are shown. FIG. 4 further shows that FOXA1 expression is absent in keratinizing squamous metaplasia of bladder urothelium, a recognized precursor to development of squamous cell carcinoma. FIGS. 5A and 5C show H&E staining of FOXA1-positive (FIG. 5B) and FOXA1-negative (FIG. 5D) samples of human SCC of the urinary bladder. Most cases (81%) of bladder SCC showed loss of FOXA1 expression.

FOXA1 Negative Tumor Cells are Proliferative.

Dual immunofluorescence of radical cystectomy patient samples depicting co-localization of FOXA1 (green) and proliferation marker KI66 (red) (data not shown). The left panel is normal adjacent tissue (NAT, top) and matched tumor from a patient with urothelial carcinoma and squamous cell carcinoma (right panel). While NAT from TCC depicted in left panel expresses FOXA1 and is KI67 negative, NAT from SCC patient displays a subpopulation of FOXA1 negative cells which are positive for KI67. Both Tumors (bottom panel) show largely mutually exclusive expression of FOXA1 and KI67. In summary, these results show FOXA1 negative cells are highly proliferative.

FOXA1 Knock-Down Results in Increased Proliferation of RT4 Tissue Recombinants.

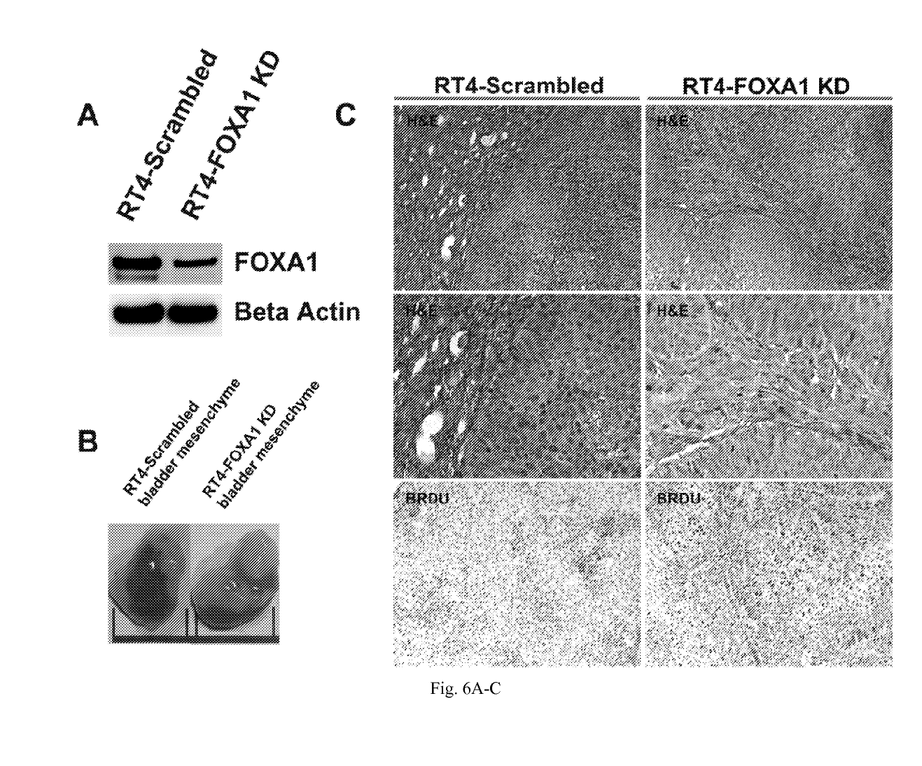

FOXA1 expression in well-differentiated RT4 human bladder cancer cells decreased via stable expression of shRNA under puromycin selection. RT4 cells stably expressing scrambled construct or FOXA1-specific shRNA were recombined with bladder mesenchyme isolated from embryonic-16 day old rats and inserted under the kidney capsule of immunocompromised mice. After three weeks, host mice were injected with BRDU and sacrificed (FIG. 6A). Tumor volume was increased in FOXA1 KD RT4 cells (FIG. 6B), and RT4-FOXA1 knock down cells showed increased incorporation of BRDU (FIG. 6C), indicating FOXA1 knock down results in increased bladder cancer cell proliferation.

Changes in FOXA1 Expression Results in Cell-Line Specific Alterations in Oncogene Expression and Cell Signaling.

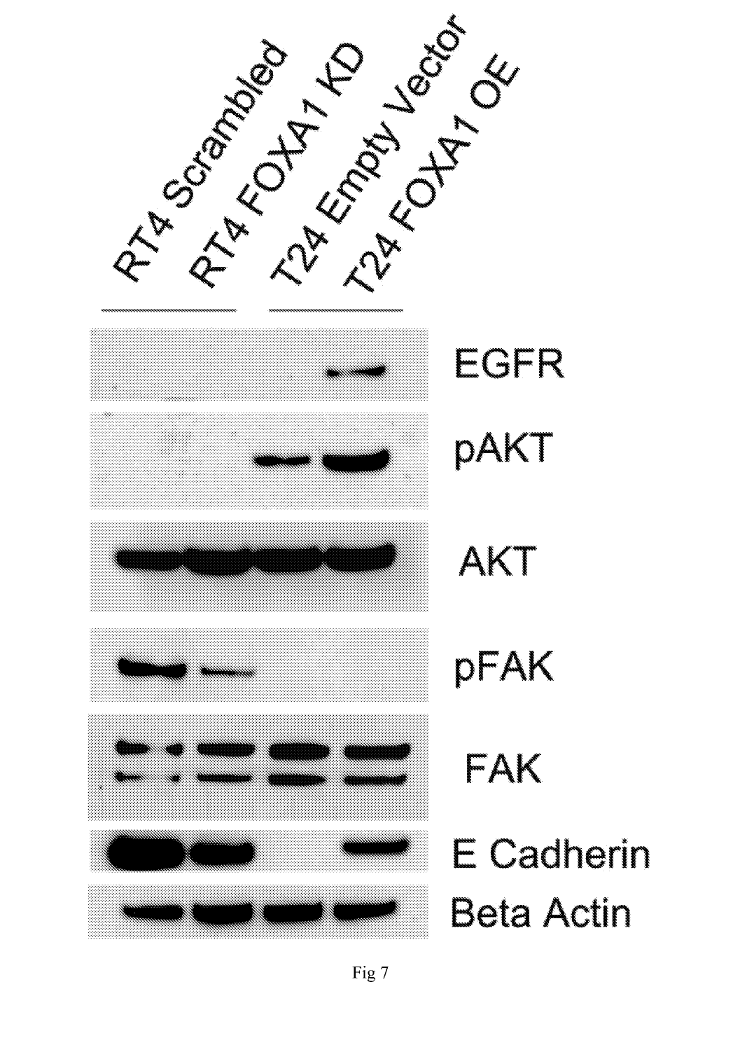

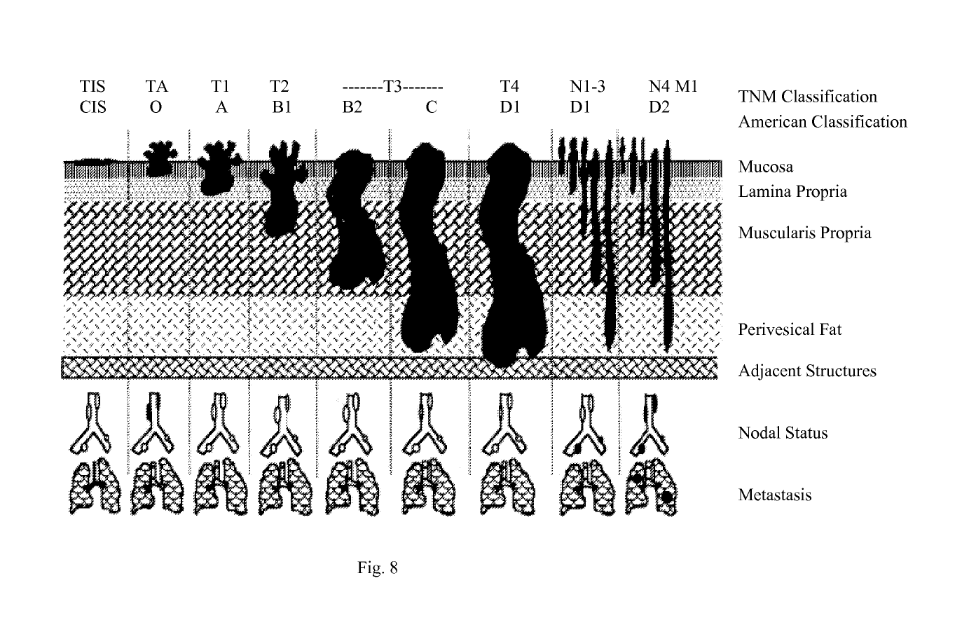

RT4 cells were engineered to express diminished FOXA1 expression (RT4 FOXA1 KD, FIG. 7, left panel), and T24 cells were engineered to ectopically overexpress FOXA1 (T24 FOXA10E, FIG. 7, right panel). Western blotting analysis showed overexpression of FOXA1 resulted in increased expression of EGFR, increased phosphorylation of AKT, and increased expression of the metastasis suppressor, E-cadherin. FOXA1 knock down resulted in decreased phosphorylation of FAK and decreased expression of E-cadherin. These results indicate FOXA1 tumor status may be useful in identifying patients who would respond to EGFR blockade and/or retinoic acid/TZD treatment.

Summary.

The inventor reports here that FOXA1 and FOXA2 are expressed in specific bladder cancer cell lines, while FOXA3 was not detected. UPK expression was correlated with FOXA1 expression in human bladder cancer cell lines and human bladder tumors (Table 1). In addition, while FOXA1 is uniformly expressed in early stage (non-muscle-invasive) bladder tumors, its expression is significantly decreased in MIBC. FOXA2 was expressed in a small number of MIBC samples. When FOXA1 staining was correlated to histological subtype, 81% of cases of squamous cell carcinoma (SCC) of the bladder were negative for FOXA1 staining compared to only 40% of transitional cell carcinomas (TCC) (Table 3). FOXA1 expression was also decreased in keratinizing squamous metaplasia, a recognized precursor to SCC. The inventors also report that decreased FOXA1 expression is associated with muscle invasion (p<0.001) and high histologic grade (p<0.001), and female gender (p<0.05). In addition, five out of 22 (23%) metastatic tumor deposits in lymph nodes from patients with MIBC were negative for FOXA1 expression. Of these 5 FOXA1-negative nodal metastases, 3 were from patients with pure SCC (Table 2). These findings support a role for FOXA1 in urothelial differentiation and provide the first evidence linking loss of FOXA1 expression with SCC and TCC subtypes of MIBC.

TABLE 1
TISSUE SOURCES AND DEMOGRAPHIC INFORMATION
Vanderbilt University Cohort

| | | |
|---|---|---|
| Gender (%) | Male | 14 (77%) |
| | Female | 4 (23%) |
| Mean age | 67 | |
| T stage (%) | Ta | 18 (100%) |
| N stage (%) | | N/A |

University of Virginia Cohort

| | | |
|---|---|---|
| Gender (%) | Male | 103 (71%) |
| | Female | 42 (29%) |
| Mean age | 67 | |
| T stage (%) | Ta | 4 (3%) |
| | T1 | 6 (4%) |
| | T2 | 47 (32%) |
| | T3 | 64 (45%) |
| | T4 | 23 (16%) |
| N stage (%) | N0 | 39 (60%) |
| | N1 or higher | 26 (40%) |
| Grade (%) | Grade 1-2 | 26 (23%) |
| | Grade 3 | 72 (63%) |
| | Grade 4 | 17 (14%) |

TABLE 2
Association of FOXA1 Staining with Gender, Tumor Stage, Grade
and Nodal Status

| | | Total | FOXA1+ (%) | FOXA1− (%) | p value |
|---|---|---|---|---|---|
| Gender | Male | 103 | 56 (54%) | 47 (45%) | p < 0.05 |
| | Female | 42 | 15 (36%) | 27 (64%)* | |
| Tumor Stage | Ta, T1 | 29 | 27 (93%) | 2 (7%) | p < 0.001** |
| | T2-T4 | 166 | 77 (46%) | 89 (54%)* | |
| Tumor Grade | G1-2 | 26 | 20 (77%) | 6 (23%) | p < 0.001 |
| | G3 | 72 | 39 (54%) | 33 (46%) | |
| | G4 | 17 | 2 (11%) | 15 (88%)* | |
| Nodal Status | N0 | 39 | 19 (49%) | 20 (51%) | p > 0.05 |
| | N1 or | 26 | 13 (50%) | 13 (50%) | |
| | greater | | | | |

TABLE 3
Histological Subtype and FOXA1 Expression

| | Total | FOXA1+ (%) | FOXA1− (%) | p value |
|---|---|---|---|---|
| SCC | 21 | 4 (19%) | 17 (81%)* | *p < 0.001 |
| TCC | 130 | 78 (60%) | 52 (40%) | |

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would he achieved. All such similar substitutes and modifications apparent to those skilled in the art are deemed to be within the spirit, scope and concept of the invention as defined by the appended claims.

### IX. REFERENCES

The following references, to the extent that they provide exemplary procedural or other details supplementary to those set forth herein, are specifically incorporated herein by reference.
U.S. Pat. No. 3,817,837
U.S. Pat. No. 3,850,752
U.S. Pat. No. 3,939,350
U.S. Pat. No. 3,996,345
U.S. Pat. No. 4,275,149
U.S. Pat. No. 4,277,437
U.S. Pat. No. 4,313,734
U.S. Pat. No. 4,366,241
U.S. Pat. No. 4,415,723
U.S. Pat. No. 4,458,066
U.S. Pat. No. 4,632,901
U.S. Pat. No. 4,683,195
U.S. Pat. No. 4,683,202
U.S. Pat. No. 4,770,853
U.S. Pat. No. 4,786,589
U.S. Pat. No. 4,800,159
U.S. Pat. No. 4,883,750
U.S. Pat. No. 5,242,974
U.S. Pat. No. 5,279,721
U.S. Pat. No. 5,384,261
U.S. Pat. No. 5,405,783
U.S. Pat. No. 5,412,087
U.S. Pat. No. 5,424,186
U.S. Pat. No. 5,429,807
U.S. Pat. No. 5,436,327
U.S. Pat. No. 5,445,934
U.S. Pat. No. 5,472,672
U.S. Pat. No. 5,527,681
U.S. Pat. No. 5,529,756
U.S. Pat. No. 5,532,128
U.S. Pat. No. 5,545,531
U.S. Pat. No. 5,554,501
U.S. Pat. No. 5,556,752
U.S. Pat. No. 5,561,071
U.S. Pat. No. 5,571,639
U.S. Pat. No. 5,593,839
U.S. Pat. No. 5,599,695
U.S. Pat. No. 5,624,711
U.S. Pat. No. 5,656,448
U.S. Pat. No. 5,658,734
U.S. Pat. No. 5,700,637
U.S. Pat. No. 5,757,994
U.S. Pat. No. 5,788,166
U.S. Pat. No. 5,795,715
U.S. Pat. No. 5,838,002
U.S. Pat. No. 5,840,873
U.S. Pat. No. 5,843,640
U.S. Pat. No. 5,843,650
U.S. Pat. No. 5,843,651
U.S. Pat. No. 5,843,663
U.S. Pat. No. 5,846,708
U.S. Pat. No. 5,846,709
U.S. Pat. No. 5,846,717
U.S. Pat. No. 5,846,726
U.S. Pat. No. 5,846,729
U.S. Pat. No. 5,846,783
U.S. Pat. No. 5,849,481
U.S. Pat. No. 5,849,486
U.S. Pat. No. 5,849,487
U.S. Pat. No. 5,849,497
U.S. Pat. No. 5,849,546
U.S. Pat. No. 5,849,547
U.S. Pat. No. 5,851,772
U.S. Pat. No. 5,853,990
U.S. Pat. No. 5,853,992
U.S. Pat. No. 5,853,993
U.S. Pat. No. 5,856,092
U.S. Pat. No. 5,858,652
U.S. Pat. No. 5,861,244
U.S. Pat. No. 5,863,732
U.S. Pat. No. 5,863,753
U.S. Pat. No. 5,866,331
U.S. Pat. No. 5,866,366
U.S. Pat. No. 5,882,864
U.S. Pat. No. 5,889,136
U.S. Pat. No. 5,900,481
U.S. Pat. No. 5,905,024
U.S. Pat. No. 5,910,407
U.S. Pat. No. 5,912,124
U.S. Pat. No. 5,912,145
U.S. Pat. No. 5,912,148
U.S. Pat. No. 5,916,776
U.S. Pat. No. 5,916,779
U.S. Pat. No. 5,919,626
U.S. Pat. No. 5,919,630
U.S. Pat. No. 5,922,574
U.S. Pat. No. 5,925,517
U.S. Pat. No. 5,928,862
U.S. Pat. No. 5,928,869
U.S. Pat. No. 5,928,905
U.S. Pat. No. 5,928,906
U.S. Pat. No. 5,929,227
U.S. Pat. No. 5,932,413
U.S. Pat. No. 5,932,451
U.S. Pat. No. 5,935,791
U.S. Pat. No. 5,935,825
U.S. Pat. No. 5,939,291
U.S. Pat. No. 5,942,391
U.S. Pat. No. 5,986,258
U.S. Pat. No. 6,004,755
U.S. Pat. No. 6,485,982
U.S. Pat. RE 35,413
Abbondanzo, Ann Diagn Pathol, 3(5):318-327, 1999.
Allred et al., Arch Surg, 125(1):107-13, 1990.
Antibodies: A Laboratory Manual, Cold Spring Harbor Laboratory, Cold Spring Harbor

Press, Cold Spring Harbor, N.Y., 1988.
Bahr et al., J. Mass Spectrom., 32:1111-1116, 1997.
Bentzley et al., Anal Chem., 68(13):2141-2146, 1996.
Bosher and Labouesse, Nat. Cell. Biol., 2:E31-E36, 2000.
Brown et al., Immunol Ser, 53:69-82, 1990.
Bucknall et al., J. Am. Soc. Mass Spectrom., 13(9):1015-1027, 2002.
Caplen et al., Gene, 252(1-2):95-105, 2000.
Caprioli et al., Anal. Chem., 69:4751, 1997.
Chaurand et al., Anal Chem., 71(23):5263-5270, 1999.
Chen and Okayama, Mol. Cell Biol., 7(8):2745-2752, 1987.
Chen et al., Nat. Biotechnol., 19:537-542, 2001.
Clark, Curr Opin Oncol., 19(3):241-7, 2007.
De Jager et al., Semin Nucl Med 23(2):165-179, 1993.
Desiderio et al., J. Mass Spectrom., 35(6):725-733, 2000.
Desiderio et al., Methods Mol. Biol., 61:57-65, 1996.
Doolittle et al., Methods Mol Biol., 109:215-237, 1999.
Duncan et al., Rapid Commun. Mass Spectrom., 7(12):1090-1094, 1993.
Elbashir et al., Genes Dev., 5(2):188-200, 2001.
Elbashir et al., Nature, 411(6836):494-498, 2001.
European Appn.—A 0 125 118
European Appn.—A 0 143 574
European Appn.—A 0 282 192
European Appn.—A 0 299 428
European Appn. 0 364 255
European Appn. 320 308
European Appn. 329 822
Faulstich et al., Anal. Chem., 69(21):4349-4353, 1997.
Fenn et al., Science, 246(4926):64-71, 1989.
Fire et al., Nature, 391(6669):806-811, 1998.
Frohman, In: PCR Protocols: A Guide To Methods And Applications, Academic Press, N.Y., 1990.
Gobom et al., Anal. Chem., 72(14):3320-3326, 2000.
Grishok et al., Science, 287:2494-2497, 2000.
Gulbis and Galand, Hum Pathol 24(12):1271-1285, 1993.
Horak et al., Rapid Commun. Mass Spectrom., 15(4):241-248, 2001.
Innis et al., Proc. Natl. Acad. Sci. USA, 85(24):9436-9440, 1988.
Jemal et al., J. Natl. Cancer Inst., 99:1384-1394, 2007.
Jespersen et al., Anal Chem., 71(3):660-666, 1999.
Jiang et al., J. Agric. Food Chem., 48:3305, 2000.
Kabarle et al., Anal. Chem. 65(20):972A-986A, 1993.
Kanazawa et al., Biol. Pharm. Bull., 22(4):339-346, 1999.
Kazmaier et al., Anesthesiology, 89(4):831-817, 1998.
Ketting et al., Cell, 99(2):133-141, 1999.
Kwoh et al., Proc. Nat. Acad. Sci. USA, 86: 1173, 1989.
Li et al., Trends Biotechnol., 18:151, 2000.
Lin and Avery, Nature, 402:128-129, 1999.
Lovelace et al., J. Chromatogr., 562(1-2):573-584, 1991.
Lynn et al., J. Mol. Evol., 48(5):605-614, 1999.
Marie et al., Anal. Chem., 72(20):5106-5114, 2000.
Miketova et al., Mol. Biotechnol., 8(3):249-253, 1997.
Mirgorodskaya et al., Rapid Commun. Mass Spectrom., 14(14):1226-1232, 2000.
Montgomery et al., Proc. Natl. Acad. Sci. USA, 95:15502-15507, 1998.
Muddiman et al., Fres. J. Anal. Chem., 354:103, 1996.
Mueller and Wold, Science 246, 780-786, 1989.
Nakamura et al., In: Handbook of Experimental Immunology (4^{th} Ed.), Weir et al., (eds). 1:27, Blackwell Scientific Publ., Oxford, 1987.
Nelson et al., Anal. Chem., 66:1408, 1994.
Nguyen et al., J. Chromatogr. A., 705(1):21-45, 1995.
Ohara et al., Proc. Nat'l Acad. Sci. USA, 86: 5673-5677, 1989.
PCT Appln. WO 88/10315
PCT Appln. WO 89/06700
PCT Appln. WO 00/44914
PCT Appln. WO 01/36646
PCT Appln. WO 01/68836
PCT Appln. WO 88/08534
PCT Appln. WO 90/07641
PCT Appln. WO 99/32619
PCT Appln. PCT/US89/01025
PCT Appln. PCT/US87/00880
Roepstorff, EXS., 88:81-97, 2000.
Sambrook et al., In: Molecular Cloning: A Laboratory Manual, 2nd Ed., Cold Spring Harbor Press, Cold Spring Harbor, N.Y., 1989.
Sengupta and Blute, Biochemistry, 45(50):15111-9, 2006.
Sharp and Zamore, Science, 287:2431-2433, 2000.
Sharp, Genes Dev., 13:139-141, 1999.
Stoeckli et al., Nat. Med., 7(4):493-496, 2001.
Tabara et al., Cell, 99(2):123-132, 1999.
Takach et al., J. Protein Chem., 16:363, 1997.
Varley et al., Cell Death & Differentiation, 2008
Villanueva et al., Enzyme Microb. Technol., 29:99, 1999.
Wagner et al., Science, 260:1510-1513, 1993.
Walker et al., Proc. Natl. Acad. Sci. USA, 89:392-396 1992.
Wang et al., Anal. Chem., 72(21):5285-5289, 2000.
Wang et al., J. Agric. Food. Chem., 47:1549, 1999.
Wang et al., J. Agric. Food. Chem., 47:2009, 1999.
Wincott et al., Nucleic Acids Res., 23(14):2677-2684, 1995.
Wittmann et al., Biotechnol. Bioeng., 72:642, 2001.
Wu et al., Anal. Chem., 70:456 A, 1998.
Wu et al., Biochem. Biophys. Res. Commun., 233(1):221-226, 1997.
Wu et al., Biochim. Biophys. Acta, 1466:315-327, 2000.
Yang et al., Proc Natl. Acad. Sci. USA, 87:9568-9572, 1990.
Zhong et al., Clin. Chem. ACTA., 313:147, 2001.
Zweigenbaum et al., Anal. Chem., 71(13):2294-300, 1999.
Zweigenbaum et al., J. Pharm. Biomed. Anal., 23(4):723-733, 2000.

1. A method of predicting or diagnosing invasive bladder cancer in a sample from a subject comprising assessing reduced FoxA1 expression, as compared to normal controls.

2. The method of claim 1, wherein assessing comprises immunologic detection or mass spectrometry.

3-4. (canceled)

5. The method of claim 1, wherein said sample is urine, semen or prostatic fluid from said subject.

6-9. (canceled)

10. The method of claim 1, wherein assessing comprises measuring expression of a FoxA1 transcript.

11-12. (canceled)

13. The method of claim 1, wherein said sample is a tumor tissue sample.

14. The method of claim 1, wherein invasive bladder cancer is muscle-invasive bladder cancer and/or metastatic bladder cancer.

15. The method of claim 1, wherein said subject previously has been diagnosed with bladder cancer as was successfully treated for said genitourinary cancer.

16. A method of monitoring the progression of and/or prognosing invasive bladder cancer in a subject comprising assessing FoxA1 level in a tumor tissue sample or the urine, semen or prostatic fluid of said subject at multiple time points, wherein an decrease in FoxA1 level over time indicates progression of said bladder cancer and a poor prognosis.

17. The method of claim 16, wherein assessing comprises immunologic detection or mass spectrometry.

18-24. (canceled)

25. The method of claim 16, wherein assessing comprises measuring expression of a FoxA1 transcript.

26-27. (canceled)

28. The method of claim 16, wherein said sample is a tumor tissue sample.

29. The method of claim 16, wherein invasive bladder cancer is muscle-invasive bladder cancer and/or metastatic bladder cancer.

30. (canceled)

31. A method of monitoring the treatment of bladder cancer in a subject comprising assessing a FoxA1 level in a tumor tissue sample or the urine, semen or prostatic fluid of said subject at multiple time points, wherein an increase in FoxA1 level over time indicates treatment efficacy.

32. The method of claim 31, further comprising altering a treatment plan based on the FoxA1 level.

33. The method of claim 31, wherein assessing comprises immunologic detection or mass spectrometry.

34-46. (canceled)

47. A method of staging a bladder cancer in a subject comprising comprising assessing FoxA1 level in a tumor tissue sample or the urine, semen or prostatic fluid of said subject and comparing said level to a predetermined level for one or more given stages of a genitourinary cancer.

48. The method of claim 47, wherein said bladder cancer is Stage 0, Stage I, Stage II, Stage III, Stage IV, or recurrent.

49. The method of claim 47, further comprising altering a treatment plan based on the FoxA1 level.

50-62. (canceled)

63. The method of claim 1, wherein assessing comprises a lateral flow ELISA assay.

64. The method of claim 1, further comprising assessing one or more patient variables, such as age, gender, extent of tumor resection, use of pre-surgery chemotherapy, or use of pre-surgery radiotherapy.

65. A method of predicting response of bladder cancer in a subject to a chemo- or radiotherapy comprising assessing FoxA1 level in a tumor tissue sample or the urine, semen or prostatic fluid of said subject at multiple time points, wherein the lower the FoxA1 level, better chance of therapeutic efficacy.
